(19) United States
(12) Patent Application Publication
(10) Pub. No.: US 2011/0104189 A1
(43) Pub. Date: May 5, 2011

(54) COMPOSITIONS OBTAINED FROM CHLORELLA EXTRACT HAVING IMMUNOMODULATING PROPERTIES

(75) Inventors: Erick Reyes Suarez, Halifax (CA); Jaroslav Kralovec, Halifax (CA); T. Bruce Grindley, Halifax (CA); Colin Barrow, Torquay (AU)

(73) Assignee: Ocean Nutrition Canada Limited, Dartmouth (CA)

(21) Appl. No.: 12/990,737

(22) PCT Filed: May 6, 2009
(86) PCT No.: PCT/IB09/06031

§ 371 Date: Jan. 17, 2011

Related U.S. Application Data
(60) Provisional application No. 61/050,859, filed on May 6, 2008.

(51) Int. Cl. A61K 39/00; C08B 37/00; A61K 38/02; A61K 31/7088; A61K 31/715; A61K 35/60; A61K 36/02; A61K 35/74; A61K 36/28; A61P 37/04; A61P 31/04; A61P 31/10; A61P 3/02

(52) U.S. Cl. 424/184.1; 536/123; 514/1.1; 514/44 R; 514/54; 424/523; 424/195.17; 424/780; 424/737

(57) ABSTRACT

The disclosed subject matter, in one aspect, relates to compounds and compositions {e.g., polysaccharides and polysaccharide complexes) and methods for providing and using such compounds and compositions. Disclosed are compositions comprising a polysaccharide or polysaccharide complex obtained from Chlorella, wherein the polysaccharide or polysaccharide complex has a molecular weight of from about 1×10³ to about 1×10⁶ Da. Also disclosed are methods of providing a polysaccharide or polysaccharide complex, comprising the steps of providing a Chlorella extract, contacting the extract with a solvent to provide a precipitate, contacting the precipitate with additional substances {e.g., a surfactant) and isolating an insoluble fraction, and size fractioning the insoluble fraction, thereby providing the polysaccharide or polysaccharide complex. Disclosed are also methods for using the disclosed polysaccharide and polysaccharide compositions.

### FIELD

Disclosed herein are polysaccharide extracts obtained from the green algae Chlorella. Also disclosed are pharmaceutical and nutritional compositions comprising the disclosed Chlorella polysaccharide extracts. Further disclosed are methods for extracting and purifying the disclosed Chlorella polysaccharide extracts. Yet further disclosed are methods for modulating an immunological response in a mammal.

### BACKGROUND

Immunotherapy has increasingly become an important approach for treating human diseases and conditions through the use of regimens designed to modulate immune responses. Immunotherapy can be particularly important in pathological conditions where the immune system becomes compromised (e.g., during cancer). Studies conducted in disease models and clinical trials demonstrate that augmenting a subject's defense mechanisms can be useful in treatment and prophylaxis against microbial infections, immunodeficiencies, cancer, and autoimmune disorders (Hadden, J. W. Immunol. Today 1993, 14, 275-280).

Immunotherapy can also have utility for promoting wound healing. During wound healing, immunotherapeutic macrophages can play a principal role by modulating cellular proliferation and new tissue formation and tissue regeneration. Macrophages also function as phagocytes, debridement agents, and stimulants for growth factors that influence the angiogenesis stage of wound repair (Wilson, K. Nurs. Crit. Care 1997, 2, 291-296).

Bacterial products (lysates and crude fractions) were among the first immunostimulants developed. These products included agents such as bacille Calmette-Guerin (BCG), Corynebacterium parvum, and lipopolysaccharide (Hadden, J. W. Immunol. Today 1993, 14, 275-280, Masihi, K. N. Int. J. Antimicrob. Agents 2000, 14, 181-191). Although these agents have had limited success due to toxicities and side-effects, many have been licensed by the United States Department of Agriculture (USDA) for immunomodulation in veterinary medicine (Van Kampen, K. R. Semin. Vet. Med. Surg. (Small Anim.) 1997, 12, 186-192).

Other immunotherapeutic agents have been developed from natural sources, chemical synthesis, and recombinant technologies. Many immunostimulants of natural origin are high molecular weight polysaccharides, glycoproteins, or complex peptides (Hadden, J. W. Immunol. Today 1993, 14, 275-280 and International Immunology Pharmacology (2006), 6, 317-333). For example, three fungal polysaccharides derived from Schizophyllum commune (schizophyllan), Lentinus edodes (lentinan) and Coriolus versicolor (krestin) are currently in clinical use in Japan as biological response modifiers (Franz, G. Planta Med. 1989, 55, 493-497.). Another polysaccharide, acemannan (isolated from Aloe vera), is licensed by the USDA for the treatment of fibrosarcoma in dogs and cats (King, G. K.; Yates, K. M.; Greenlee, P. G.; Pierce, K. R.; Ford, C. R.; McAnalley, B. H.; Tizard, I. R. J. Am. Animal Hosp. Assoc. 1995, 31:439-47). There are a few small molecular weight immunostimulants derived from natural products such as the glycosphingolipid KRN-7000. A clinical trial using KRN-7000 as an immunostimulant for treatment of solid tumors is currently in progress (Natori, T.; Motoki, K.; Higa, T.; Koezuka Y., In “Drugs from the Sea;” Fusetani, N., Ed.; Karger: New York, 2000; pp 86-97.). Several immunostimulants of synthetic origin also have been developed that include compounds like isoprinosine and muramyl peptides (Masihi, K. N. Int. J. Antimicrob. Agents 2000, 14, 181-191). Recently, a number of other immunomodulators of endogenous origin have been developed using recombinant technologies, and many of these have gained FDA approval. These agents include colony-stimulating factors, interferons and recombinant proteins (Frank, M. O.; Mandell, G. L. Immunomodulators In Principles and Practice of Infectious Diseases, Ch. 33, 4th ed.; Mandell, G. L., Bennett, J. E., Dolin, R., Eds.; Churchill Livingstone: New York, 1995; pp 450-458.). These compounds, however, often have short half-lives and it can be difficult to determine optimal dosage and appropriate combinations.

Recently, immunotherapeutic agents derived from microalgae have been receiving increasing interest. Microalgae have been used as nutrient-dense food sources since ancient times, and historical records indicate that microalgae such as Spirulina platensis were consumed by tribes around Lake Chad in Africa and by the Aztecs living near Lake Texcoco in Mexico. Many are increasingly interested in the commercial production of food-grade microalgae for human consumption and as feed for livestock. Among the various microalgae that have been explored for their commercial potential, Spirulina species, Chlorella species, and Aphanizomenon flos-aquae (AFA) are three major types that have been successfully produced.

Chlorella is edible, unicellular green microalgae believed to have many desirable immunotherapeutic properties and has been called a sun-powered supernutrient. It is known that Chlorella can be useful in wound healing, detoxification, constipation relief, and growth stimulation. A number of studies have also indicated that Chlorella can have desirable immunostimulatory properties, both in vitro and in vivo.

Chlorella can be found in both fresh water and marine water. Species of the Chlorella genus exhibit striking diversity of physiological and biochemical properties (Kessler, E. “Phycotalk” 1989, 1:141-153; V. Rastogi Publ., New Delhi, India). Chlorella produces little cellulose and other indigestible cell wall material, and hence has been extensively investigated as a possible new source of food, especially as feedstock (Lee, Robert E. “Phycology” 2^{nd} edition; 1989, page 281; Cambridge University Press).

It is believed that Chlorella has the highest content of chlorophyll of any known plant. Chlorella also contains vitamins, minerals, dietary fiber, nucleic acids, amino acids, enzymes, and other biological substances. It contains more than about 9% fats; of this 9%, polyunsaturated fatty acids represent about 82%. The vitamin content comprises provitamin A, vitamins B₁, B₂, B₆, niacin, B₁₂, biotin, vitamin C, vitamin K, pantothenic acid, folic acid, choline, lipoic acid, ionositol, and PABA. Minerals present in Chlorella include P, K, Mg, S, Fe, Ca, Mn, Cu, Zn and Co.

Aqueous extracts of Chlorella have been used for nutritional and other health benefits. Such extracts were introduced as health foods in 1977 when processes were developed that made Chlorella more easily digestible. The Taiwan Chlorella company is the world's largest supplier of Chlorella, and sells the product to Asia, Europe and North America, under the following brand names: ALGEA™, BIO-REURELLA™, GREEN GEM™ GREEN BOOST™, GREEN NATURE™, GREEN POWER™, JOYAU VERT™ and NATURAL BOOST™.

A number of Chlorella extracts are also available commercially, including products by Swiss Herbal™ and Nature's Way™. The Swiss Herbal product is identified as pure Chlorella broken cells containing Protein 61%, Carbohydrate 21.1%, Fat 11.0%, Chlorophyll 2.87%, RNA 2.94% and DNA 0.28%.

Oral administration of Chlorella pyrenoidosa has been correlated with enhanced natural killer cell activity and increased granulocyte-macrophage progenitor cells in mice infected with Listeria monocytogenes. For all these effects, however, the active components have not been conclusively established. A number of polysaccharides that possess biological activity have been identified from Chlorella species. In U.S. Pat. No. 4,533,548 an acidic polysaccharide was isolated from Chlorella pyrenoidosa that exhibits antitumor and antiviral activity. The glycosyl composition for this polysaccharide was mostly rhamnose, with minor amounts of galactose, arabinose, glucose and glucuronic acid. Another polysaccharide, isolated from marine Chlorella minutissima, reported in U.S. Pat. No. 4,831,020, appears to have tumor growth-inhibiting effects.

In U.S. Pat. No. 4,786,496, the lipid fraction (glycolipid portion) of marine Chlorella species displayed antitumor properties. Several glycoproteins have also been isolated from Chlorella species. For example, U.S. Pat. No. 4,822,612 reported a 45,000 dalton glycoprotein that has anticancer effects. Various other glycoproteins and glyceroglycolipids that can have immunopotentiating and antitumor properties also have been reported in the scientific literature.

U.S. Pat. No. 5,585,365 discloses that an antiviral polysaccharide with a molecular weight between 250,000 and 300,000 daltons was isolated from Spirulina species using hot water extraction. This polysaccharide is composed of rhamnose, glucose, fructose, ribose, galactose, xylose, mannose, glucuronic acid and galacturonic acid. A number of other low molecular weight polysaccharides that range between 12,600 and 60,000 daltons recently have been isolated from Spirulina species.

Although current Chlorella based immunostimulants show promise, there is still a need to identify and develop potent agents and increase the arsenal of available drugs for immunotherapy.

### SUMMARY

In accordance with the purposes of the disclosed materials, compositions, articles, devices, and methods, as embodied and broadly described herein, the disclosed subject matter, in one aspect, relates to compounds and compositions (e.g., polysaccharides and polysaccharide complexes) and methods for providing and using such compounds and compositions. Disclosed herein are compositions that comprise a polysaccharide or polysaccharide complex obtained from Chlorella, wherein the polysaccharide or polysaccharide complex has a molecular weight of from about 1×10³ to about 1×10⁶ Da.

Also disclosed herein are methods of providing a polysaccharide or polysaccharide complex, comprising the steps of providing a Chlorella extract, contacting the extract with a solvent to provide a precipitate, contacting the precipitate with additional substances (e.g., a surfactant) and isolating an insoluble fraction, and size fractioning the insoluble fraction, thereby providing the polysaccharide or polysaccharide complex. Disclosed are also methods for using the disclosed polysaccharide and polysaccharide compositions.

It has now been found that polysaccharides from microalgae have not been identified in detail as those from bacteria, yeasts, and plants. Disclosed herein are phosphoglycans obtained from microalgae origin containing a glycosyl phosphate structure. Side chains of α-Manp-(1-PO₃H→ units is a structural feature that resemble some yeasts phosphoglycans structures. Further disclosed is the presence of methylated phosphosaccharide units, 3-O-methyl α-Manp-(1-PO₃H→ units.

Additional advantages will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 depicts an example of a flow-chart providing an example of a procedure that can be used to obtain fraction A-P-8.

FIG. 2 depicts a size-exclusion chromatography graph of fraction A-P fractionation on Sephadex G-100.

FIG. 3 depicts an anion exchange chromatography graph of fraction A-P-1 fractionation on Q-Sepharose Fast Flow.

FIG. 4 depicts the 202.5 MHz ³¹P NMR spectrum of fraction A-P-8-deO in D₂O at 27° C.

FIG. 5 depicts the 125 MHz ¹³C DEPTQ 135 NMR spectrum of fraction A-P-8-deO in D₂O at 27° C.

FIG. 6 depicts a size exclusion chromatography graph of fraction A-P-8-deO-deP fractionation in BioGel P-2 and the fractions found wherein, A-P-8-deO-deP-1, A-P-8-deO-deP-2, and A-P-8-deO-deP-3.

FIG. 7 depicts the 125 MHz ¹³C DEPTQ 135 NMR spectrum of fraction A-P-8-deO-deP-3 in D₂O at 27° C.

FIG. 8 depicts the 125 MHz ¹³C DEPTQ 135 NMR spectrum of fraction A-P-8-deO-deP-2 in D₂O at 27° C.

FIG. 9 depicts the 125 MHz ¹³C DEPTQ 135 NMR spectrum of fraction A-P-8-deO-deP-1 in D₂O at 27° C.

FIG. 10 depicts the 500.1 MHz ¹H NMR spectrum of fraction A-P-8-deO-deP-1 in D₂O at 50° C.

FIG. 11 depicts the 800 MHz TOCSY spectrum of fraction A-P-8-deO-deP-1 in D₂O at 60° C.

FIG. 12 depicts the 800 MHz COSY spectrum of fraction A-P-8-deO-deP-1 in D₂O at 60° C.

FIG. 13 depicts the ¹H ¹³C HSQC spectrum at 800 MHz of fraction A-P-8-deO-deP-1 in D₂O at 60° C.

FIG. 14 depicts the ¹H ¹³C HMBC spectrum at 500 MHz of fraction A-P-8-deO-deP-1 in D₂O at 27° C. using a 60 ms mixing time.

FIG. 15 depicts the 800 MHz NOESY spectrum fraction A-P-8-deO-deP-1 in D₂O at 60° C. using a 200 ms mixing time.

FIG. 16 depicts two sequences of regular substitution patterns of glucose on galactoses that are consistent with the H-4-s/H-1s NOE correlations at 4.23/4.73 ppm and 4.26/4.73 ppm; (a) regular alternating; b) on blocks of two adjacent galactoses.

FIG. 17 depicts the 500.1 MHz ¹H NMR spectrum of fraction A-P-8-deO in D₂O at 27° C.

FIG. 18 depicts the ¹H ¹³C HSQC spectrum at 800 MHz fraction A-P-8-deO in D₂O at 60° C.

FIG. 19 depicts the 800 MHz COSY spectrum of fraction A-P-8-deO in D₂O at 60° C.

FIG. 20 depicts the ¹H ¹³C HMBC spectrum at 800 MHz of fraction fraction A-P-8-deO in D₂O at 50° C. using a 60 ms mixing time.

FIG. 21 depicts the 800 MHz NOESY spectrum of fraction A-P-8-deO in D₂O at 60° C. using a 150 ms mixing time.

FIG. 22 depicts the ¹H ³¹P HSQC spectrum of fraction A-P-8-deO in D₂O at 27° C. with an evolution delay adjusted to 8 Hz.

FIG. 23 depicts a portion of the 125 MHz ¹³C DEPTQ 135 NMR spectra of the de-O-acetylated fraction A-P-8-deO (top) and of the intact fraction A-P-8 (bottom) showing the effects of de-O-acetylation, with the more noticeable changes on signals shapes and intensities being highlighted

FIG. 24 depicts a portion the ¹³C DEPTQ 135 NMR spectrum (125 MHz) of the de-O-acetylated fraction A-P-8-deO (top) and of the intact fraction A-P-8 (bottom) showing the effects of de-O-acetylation at O-2 on the peak shapes and intensities of the C-1 and the C-3 of galactoses.

FIG. 25 is a bar graph showing stimulation of peritoneal macrophages of murine origin by fractions derived from fractionation of Chlorella pyrenoidosa.

FIGS. 26-34 depict examples of a polysaccharide or polysaccharide complex obtained from Chlorella according to the present disclosure.

### DETAILED DESCRIPTION

The materials, compounds, compositions, articles, and methods described herein can be understood more readily by reference to the following detailed description of specific aspects of the disclosed subject matter and the Examples included herein and to the Figures.

Before the present materials, compounds, compositions, articles, and methods are disclosed and described, it is to be understood that the aspects described below are not limited to specific synthetic methods or specific reagents, as such can, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### DEFINITIONS

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a compound” includes mixtures of two or more such compounds, reference to “an agent” includes mixtures of two or more such agents, reference to “the moiety” includes mixtures of two or more such moieties, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed then “less than or equal to” the value, “greater than or equal to the value” and possible ranges between values are also disclosed, as appropriately understood by the skilled artisan. For example, if the value “10” is disclosed, then “less than or equal to 10” as well as “greater than or equal to 10” is also disclosed. It is also understood that throughout the application, data is provided in a number of different formats, and that this data represents endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

“Quaternary ammonium surfactant,” as used herein, means any nitrogen compound wherein at least one nitrogen atom is bonded to four atoms (e.g., a cationic nitrogen) corresponding to the following general structure,

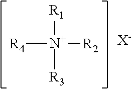

wherein at least one of R₁, R₂, R₃ and R₄ is any substituent comprising from 1 to 26 carbon atoms, and wherein X⁻ can be any suitable anion (e.g., Br⁻, Cl⁻, F⁻, I⁻, CO₃²⁻, HCO₃⁻, OH⁻, ClO₃⁻, ClO₄⁻, ClO₂⁻, ClO⁻, CrO₄²⁻, Cr₂O₇²⁻, IO₃⁻, NO₃⁻, NO₂⁻, PO₄³⁻, HPO₄²⁻, H₂PO₄⁻, MnO₄⁻, PO₃³⁻, SO₄²⁻, S₂O₃²⁻, HSO₄⁻, SO₃²⁻, HSO₃⁻, other inorganic anions, other organic anions, and the like).

Suitable quaternary ammonium compounds include (C₁₂-C₁₄ alkyl)(C₁-C₂ dialkyl)-benzyl ammonium salts, N—(C₁₂-C₁₈ alkyl)heteroaryl ammonium salts, and N—[(C₁₂-C₁₄ alkyl)(C₁-C₂ dialkyl)]heteroarylalkylene ammonium salts. Non-limiting examples of the (C₁₂-C₁₄ alkyl)(C₁-C₂ dialkyl)benzyl ammonium salts include (C₁₂-C₁₄ alkyl)dimethyl-benzyl ammonium chloride, (C₁₂-C₁₄ alkyl)dimethylbenzyl ammonium bromide, and (C₁₂-C₁₄ alkyl)dimethylbenzyl ammonium hydrogen sulfate. Non-limiting examples of the N—(C₁₂-C₁₈ alkyl)heteroaryl ammonium salts include cetyl pyridinium chloride, cetyl pyridinium bromide, and cetyl pyridinium hydrogen sulfide. For the N—(C₁₂-C₁₈ heteroaryl ammonium salts other anions can be used.

Further examples of quaternary ammonium compounds suitable for use include cetyltrimethylammonium chloride, stearyltrimethylammonium chloride, isostearyltrimethyl-ammonium chloride, lauryltrimethylammonium chloride, behenyltrimethyl-ammonium chloride, octadecyltrimethylammonium chloride, cocoyltriinethylammonium chloride, cetyltrimethylammonium bromide, stearyltrimethylammonium bromide, lauryl-trimethyl-ammonium bromide, isostearyllauryldimethylammonium chloride, dicetyldimethyl-ammonium chloride, distearyldimethylammonium chloride, dicocoyldimethylammonium chloride, γ-gluconamidopropyldimethylhydroxyethylammonium chloride, di-[polyoxyethylene(2)]oleylmethylammonium chloride, dodecyldimethylethylammonium chloride, octyldihydroxyethylmethylammonium chloride, tri[polyoxyethylene(5)]-stearylammonium chloride, polyoxypropylenemethyldiethylammonium chloride, lauryl-dimethyl(ethylbenzyl)ammonium chloride, behenamidopropyl-N,N-dimethyl-N-(2,3-dihydroxypropyl)ammonium chloride, tallowedimethylammoniopropyltrimethylammonium dichloride, and benzalconium chloride.

“Subject,” as used herein, means an individual. In one aspect, the subject is a mammal such as a primate, and, in another aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

By the term “effective amount” of a compound or composition as provided herein is meant a nontoxic but sufficient amount of the compound to provide the desired utility, for example to reduce, inhibit, prevent, or otherwise modulate an immune response. As will be pointed out below, the exact amount required will vary from subject to subject, depending on the species, age, body weight, general health, sex, diet, and general condition of the subject, the severity of the condition or disease that is being treated, the particular compound used, its mode of administration, the duration of the treatment, drugs used in combination or coincidental with the specific composition employed, and like factors well known in the medical arts. Thus, it is not possible to specify an exact “effective amount”; however, an appropriate effective amount can be determined by one of ordinary skill in the art using only routine experimentation. For example, it is well within the skill of the art to start doses of a composition at levels lower than those required to achieve the desired therapeutic effect and to gradually increase the dosage until the desired effect is achieved. One can also evaluate the particular aspects of the medical history, signs, symptoms, and objective laboratory tests that are known to be useful in evaluating the status of a subject in need of attention for the treatment of a disease. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and/or knowledge of the normal progression of the disease in the general population or the particular individual: 1) a subject's physical condition is shown to be improved, 2) the progression of the disease or condition is shown to be stabilized, or slowed, or reversed, or 3) the need for other medications for treating the disease or condition is lessened or obviated, then a particular treatment regimen will be considered efficacious. If desired, the effective daily dose can be divided into multiple doses for purposes of administration. Consequently, single dose compositions can contain such amounts or submultiples thereof to make up the daily dose. The dosage can be adjusted by the individual physician or the subject in the event of any counterindications. Dosage can vary, and can be administered in one or more dose administrations daily, for one or several days. Guidance can be found in the literature for appropriate dosages for given classes of pharmaceutical products.

The term “effective amount” of an immunomodulator refers to an amount of an immunomodulator sufficient to enhance a subject's defense mechanism. This amount can vary to some degree depending on the mode of administration. More than one immunomodulator can also be used (e.g., Chlorella extract in combination with Echinacea). The exact effective amount necessary can vary from subject to subject, depending on the species, age and general condition of the subject, the severity of the condition being treated, the mode of administration, etc. The appropriate effective amount can be determined by one of ordinary skill in the art using only routine experimentation or prior knowledge in the immunomodulator art.

The term “pharmaceutically acceptable” means a material that is not biologically or otherwise undesirable, i.e., the material can be administered to an individual along with a selected Chlorella polysaccharide, for example, without causing any undesirable biological effects or interacting in a deleterious manner with any of the other components of the pharmaceutical composition in which it is contained.

The term “pharmaceutically acceptable derivative” refers to any homolog, analog, or fragment corresponding to the compounds disclosed herein, which modulate an immune response of subject.

As used herein, and without limitation, the term “derivative” is used to refer to any compound which has a structure derived from the structure of the compounds disclosed herein and whose structure is sufficiently similar to those disclosed herein and based upon that similarity, would be expected, by one skilled in the art, to exhibit the same or similar activities and utilities as the claimed compounds.

The term “alkyl” and “aliphatic” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. The alkyl group can also be substituted or unsubstituted. The alkyl group can be substituted with one or more groups including, but not limited to, alcohol, alkyl, halogenated alkyl, alkoxy, alkenyl, alkynyl, aryl, heteroaryl, aldehyde, amino, carboxylic acid, ester, halide, hydroxamate, hydroxy, ketone, nitro, silyl, sulfo-oxo, sulfonyl, sulfone, sulfoxide, or thiol, as described below. The term “halogenated alkyl” specifically refers to an alkyl group that is substituted with one or more halide, e.g., fluorine, chlorine, bromine, or iodine. The term “higher aliphatic” can refer to an aliphatic compound of from about 6 to 24 carbon atoms.

Disclosed herein are nucleic acid based materials. Examples of nucleic acids described herein include, but are not limited to, DNA, such as cDNA, and RNA, such as mRNA. The disclosed nucleic acids are made up of, for example, nucleotides, nucleotide analogs, or nucleotide substitutes. Non-limiting examples of these and other molecules are discussed herein. It is understood that, for example, when a vector is expressed in a cell, that the expressed mRNA will typically be made up of A, C, G, and U.

A “nucleotide” as used herein is a molecule that contains a base moiety, a sugar moiety, and a phosphate moiety. Nucleotides can be linked together through their phosphate moieties and sugar moieties creating an internucleoside linkage. The term “oligonucleotide” is sometimes used to refer to a molecule that contains two or more ucleotides linked together. The base moiety of a nucleotide can be adenine-9-yl (A), cytosine-1-yl (C), guanine-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide that contains some type of modification to the base, sugar, and/or phosphate moieties. Modifications to nucleotides are well known in the art and would include, for example, 5-methylcytosine (5-me-C), 5 hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but are linked together through a moiety other than a phosphate moiety. Nucleotide substitutes are able to conform to a double helix type structure when interacting with the appropriate target nucleic acid.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more (e.g., referred to as “disubstituted,” “trisubstituted,” and the like) and the same or different for appropriate organic compounds. For purposes of this disclosure, the heteroatoms, such as nitrogen and oxygen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. Also, as used herein “substitution” or “substituted with” is meant to encompass configurations where one substituent is fused to another substituent. For example, an aryl group substituted with an aryl group (or vice versa) can mean that one aryl group is bonded to the second aryl group via a single sigma bond and also that the two aryl groups are fused, e.g., two carbons of one alkyl group are shared with two carbons of the other aryl group.

As used herein, the term “immunomodulator” refers to an agent which is able to modulate an immune response. The term “modulate” refers to the ability of an agent (e.g., an immunomodulator) to regulate an immune system. Modulate, as used herein, can refer to a process by which an agent elevates or reduces an immune response. Modulate refers to the ability of an agent to regulate an immune response either directly or indirectly (e.g., an immunomodulator can regulate a mechanism that occurs during an immune response, thereby regulating the overall immune response). Modulate can refer to a process by which an agent substantially inhibits, stabilizes, or prevents an increased immune response when an immune response would otherwise increase. Modulate can also refer to a process by which an agent substantially stabilizes, enhances, or maintains an immune response when an immune response would otherwise decrease. Such modulation, for example, can be useful in the treatment of various autoimmune diseases, among other diseases. Modulate can also refer to a process by which an agent induces an immune response or substantially prevents an immune response.

The term “treatment” as used herein covers any treatment of a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixtures.

Reference will now be made in detail to specific aspects of the disclosed materials, compounds, compositions, articles, and methods, examples of which are illustrated in the accompanying Examples and Figures.

### Materials and Compositions

Disclosed herein are materials, compounds, compositions, and components that can be used for, can be used in conjunction with, can be used in preparation for, or are products of the disclosed methods, devices, and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a composition is disclosed and a number of modifications that can be made to a number of components or residues of the composition are discussed, each and every combination and permutation that are possible are specifically contemplated unless specifically indicated to the contrary. Thus, if a class of components or residues A, B, and C are disclosed as well as a class of components or residues D, E, and F, and an example of a combination compound A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Nomenclature recommended by the International Union of Pure and Applied Chemistry (IUPAC) is used to reference the various polysaccharides, polysaccharide complexes, oligosaccharides, and saccharides disclosed herein, unless specifically stated to the contrary. Recommendations made by the IUPAC are outlined in the following publications: “Polysaccharide nomenclature. Recommendations 1980,” Arch. Biochem. Biophys., 1983, 220, 330-332; Eur. J. Biochem., 1982, 126, 439-441; J. Biol. Chem., 1982, 257, 3352-3354; Pure Appl. Chem., 1982, 54, 1523-1526, which are hereby incorporated into this specification by reference.

Immunomodulating compositions obtained from Chlorella have been disclosed in U.S. Pat. Nos. 6,551,596, 6,974,576, and 6,977,076, and U.S. Patent Publication No. 2007/0264271, each of which is hereby incorporated into this disclosure in its entirety. The presently disclosed subject matter relates to methods and novel compositions related to Chlorella and Chlorella extract. The compositions disclosed herein are contemplated for use as, inter alia, immunomodulators. Methods are disclosed herein for providing the compositions disclosed herein including the steps of providing a Chlorella extract, providing a precipitate from the extract, contacting the precipitate with a substance so as to isolate an insoluble fraction, and size fractionating the insoluble fraction by using a molecular weight fractionation, thereby providing the polysaccharide or polysaccharide complex.

Compositions obtained from the methods disclosed herein are also disclosed. Specifically disclosed are compositions comprising a polysaccharide or polysaccharide complex obtained from Chlorella, wherein the polysaccharide or polysaccharide complex has a molecular weight of from about 1×10³ to about 1×10⁵ Daltons. Methods for using the disclosed compositions (e.g. as immunomodulators, pharmaceutical agents, nutritional supplements, etc.) are also disclosed.

The compositions disclosed herein can also be obtained from C. minutissima, C. marina, C. salina, C. vulgaris, C. anitrata, C. antarctica, C. autotrophica, C. regularis, C. ellipsoidea, or mixtures thereof.

### Chlorella

Disclosed herein are extracts derived from Chlorella. Species of the Chlorella genus from which extracts can be obtained comprise, without limitation, minutissima, marina, salina, pyrenoidosa, vulgaris, anitrata, antarctica, autotrophica, regularis, and any combination thereof, among others. Many of these species and other species are described in the “World Catalog of Algae,” 2^{nd} Ed, pp. 58-74; Miyachi et al. (Eds); 1989; Japan Scientific Societies Press.

Mutant strains of Chlorella, either naturally occurring or artificially produced, for example by irradiation (e.g. ultraviolet, X-ray), chemical mutagenesis or by site-directed mutagenesis, are also contemplated for use with the disclosed subject matter. In one example, Chlorella pyrenoidosa and its variants can be used. In another example, Chlorella ellipsoidea and its variants can be used.

Cultivation of Chlorella can be carried out by methods known in the art using suitable media and culture conditions (see, for example, White and Barber, Biochimica Biophysica Acta, 1972, 264, 117-128). It should be appreciated that polysaccharide production can be influenced by physiological and metabolic manipulation of Chlorella cultures. Moreover, composition of the growth media can influence growth rates leading to changes in Chlorella cell wall thicknesses. It should also be appreciated that genes responsible for growth present in Chlorella can be up- or down-regulated. Methods to transform eukaryotic algae (e.g., Chlorella) are known (see, for example, U.S. Pat. No. 6,027,900) as well as methods to select algal mutants (see, for example, U.S. Pat. No. 5,871,952); such methods are contemplated for use with the disclosed subject matter. Thus, by selection under various conditions, variants of biopolymer immunomodulators from Chlorella can be manufactured.

Crude Chlorella extracts can be prepared by methods known in the art, including hot water extraction of cultured cells or spray dried cells (U.S. Pat. Nos. 4,831,020 and 5,780,096) and solvent extraction methods (White and Barber, Biophys. Biochim. Acta, 1972, 264:117-128; U.S. Pat. No. 3,462,412). Crude extracts can also be obtained from the Taiwan Chlorella company. Other extraction methods are described in more detail in U.S. Pat. No. 6,551,596, U.S. Pat. Nos. 6,974,576, and 6,977,076, which have been incorporated by reference herein before.

In one example, the crude Chlorella extract can be prepared from spray-dried Chlorella cells by treating the cells with aqueous media, preferably water or weak solutions of organic acids, such as acetic acid, ascorbic acid, benzoic acid, citric acid, lactic acid, maleic acid, propionioc acid, sorbic acid, succinic acid, etc., or any combination thereof, under gentle agitation. The extraction process can be executed at various temperatures ranging from about 0 to about 100° C., or from about 50 to about 90° C. In a specific example, Chlorella cells (e.g. Chlorella pyrenoidosa) can be suspended in distilled water and extracted at least about 80° C. The extraction period can be carried out over any suitable time period. For example, extraction periods ranging from about 0 to about 5 hours can be used. A specific example includes an extraction period lasting about 1 hour.

The residual cells and the cell debris can be separated by centrifugation with a relative centrifugal force (RCF) of about 150 to about 10,000 g. The time necessary to complete this step can be related to the centrifugal force; for example, about 20 minutes can be sufficient at 10,000 g. The supernatant can then be micro-filtered. Alternatively, filtration can be used to remove whole cells and debris, in which case use of a series of filters starting from coarse, through medium, and ending with micro-filtration, can be useful. Cross-flow filtration or vibrating membrane technology can be used to reduce fouling. It should be appreciated that filtration can be particularly sensitive to temperature and extraction time period.

After centrifugation or filtration, the supernatant (or filtrate) can be concentrated and/or dried to obtain products in dry form. Drying can be achieved by lyophilization, supernatant evaporation in vacuo, cold airflow, or by spray-drying. The volume of the extract can also be reduced first (to 10-50%, for example), and then the active materials can be precipitated from the solution with suitable precipitants, such as ethanol or ammonium sulfate.

Various other Chlorella products (some of which are available pre-processed) can also be used with the disclosed subject matter. Commercially available Chlorella products, for example, can be used. Examples of commercially available formulations and products contemplated for use with the disclosed subject matter comprise, inter alia, RESPONDIN™ (Ocean Nutrition Canada Limited, Dartmouth, Nova Scotia, Canada), SUN CHLORELLA™ (Sun Chlorella, Torrance, Calif., U.S.A.), and CHLORENERGY™ (Chlorella Industry Co., Ltd, Chikugo City, Japan), and any combination thereof.

The Chlorella extracts can comprise various different percentages of polysaccharide and polysaccharide complexes as a fraction of the total Chlorella-derived content of the extract. The percentage can be at least 24% (w/w), at least 26% (w/w), at least 28% (w/w), at least 30% (w/w), at least 35% (w/w), at least 40% (w/w), at least 45% (w/w), at least 50% (w/w), or at least 60% (w/w).

It is understood that the Chlorella and Chlorella compositions disclosed herein can be used in combination with the various compositions disclosed herein, methods disclosed herein, products disclosed herein, and applications of the disclosed subject matter.

### Fractionation Methods

Crude Chlorella extract derived from the aforementioned methods disclosed herein and methods alike can be further processed and fractioned to retrieve desired components of the extract, which are referred to herein as a “fraction” or “fractions.” Crude Chlorella extract, for example, can be suspended in a polar medium and precipitation can be used to further separate the crude extract. Any suitable water soluble organic solvent that induces precipitation is contemplated for use with the disclosed subject matter. Examples comprise, inter alia, methanol, ethanol, propanol, acetone, ethylene glycol, tetrahydrofuran, isopropanol, ammonium sulphate, and any combination thereof. A specific example comprises the selection of about 95% ethanol for use as a precipitation solvent. Any suitable volume of precipitating solvent can be used, and, in general, can depend on the size of the crude extract desired for further processing.

Following precipitation, or another crude fractioning technique, additional processes can be used to treat the crude extract. Suspended precipitates of the crude extract, for example, can be centrifuged, dialyzed, and/or freeze-dried to give a substantially dry precipitate. Additionally, precipitates can be decolorized using methods well known in the art. For example, a precipitate can be decolorized by stirring a suspension or solution of precipitate with a mixture of a decolorizing agent (e.g., 2-chloroethanol). A specific example comprises the use of a mixture (e.g., 2:1 mixture) of CH₃Cl:CH₃OH to decolorize a precipitate. A decolorized mixture can then be treated further to process the precipitate to a desired quality. Decolorized mixtures can, for example, be dialyzed and/or freeze-dried to produce substantially dry precipitates.

The polysaccharide and polysaccharide complexes can be further purified and isolated by removal of non-polysaccharide components. Such non-polysaccharide components include nucleic acids (e.g., DNA, RNA) and protein. One method of removal is the use of digestion enzymes to cleave the non-polysaccharide components, followed by size fractionation to remove the cleaved products as described in U.S. Pat. No. 6,551,596. Digestion enzymes include pronase, ribonuclease, DNase and proteases, as are well known in the art and described in various text books, one example of which is Maniatis et al., Molecular Cloning: A Laboratory Manual (1982) Cold Spring Harbor Laboratory Press, Cold Spring Harbor, N.Y. Proteases useful for digestion of unassociated proteins include: endo- and exopeptidases, pronase, serine proteases such as trypsin, chymotrypsin and subtilisin, thiol proteases such as papain, and calcium-requiring proteases such as thermolysin.

Alternatively, non-polysaccharide components can be removed by affinity chromatography, for example by use of DNA- or RNA-binding matrices (Maniatis et al., 1982). Another option is to purify the polysaccharide and polysaccharide complexes away from the contaminating components by use of polysaccharide binding matrices such as lectins. In another example, the extracts disclosed herein can be treated with glycolytic enzymes under conditions and for a length of time sufficient to effect cleavage of: (i) three or more α-1,4-linked D-glucose units; (ii) α-1,4-linked glucosides; (iii) α-1,4-linked galactosides; or (iv) α-1,4-linked D-glucose. After such a treatment, compositions can retain their immunomodulating activity.

Fractions obtained by precipitation or other methods used on crude Chlorella extract can be further fractionated and purified. Fractions can be treated, for example, with a surfactant to achieve further fractionation. Surfactants contemplated for use with the disclosed subject matter comprise quaternary ammonium surfactants as disclosed herein before (e.g., ammonium lauryl sulfate, cetyltrimethylammonium bromide (CTAB), hexadecyltrimethylammonium bromide, other alkyltrimethylammonium salts). Surfactants contemplated for use with the disclosed subject matter also comprise, inter alia, sodium dodecyl sulfate (SDS), other alkyl sulfate salts, sodium laureth sulfate, (sodium lauryl ether sulfate), alkyl benzene sulfonate, cetylpyridinium chloride (CPC), polyethoxylated tallow amine (POEA), benzalkonium chloride (BAC), benzethonium chloride (BZT), dodecyl betaine, dodecyl dimethylamine oxide, cocamidopropyl betaine, coco ampho glycinate, and any combination thereof. Aqueous solutions of the aforementioned surfactants can also be used to achieve further fractionation. Any appropriation weight-to-volume (w/v), or weight-to-weight (w/w) ratio of surfactant and water can be used. Examples include ratios of about 1, 2, 3, 4, 5, 6, 7, 8, 9, 10, 11, 12, 13, 14, 15, 16, 17, 18, 19, 20, 21, 22, 23, 24, 25, 30, 40, 50, 60, and about 90 w/v (surfactant/water). After treatment of an appropriate fraction with a surfactant, a mixture can be further processed, precipitated, filtered, dialyzed, and/or freeze-dried to yield an appropriate sub-fraction.

Size-fractionation can be used in accordance with the methods and compositions disclosed herein. Size-fractionation, for example, can be used to further separate Chlorella extract, components of Chlorella extract, fractions and sub-fractions of Chlorella extract, precipitates of Chlorella extract, etc. Size fractionation can be accomplished by any method known in the art, including size exclusion chromatography, sedimentation analysis (e.g., gradient centrifugation, and ultra-filtration.)

Size fractionation to obtain the suitable fractions and sub-fractions of Chlorella extract can be based on principles of molecular sieving. Such basic principles of size exclusion chromatography are well known to those in the art and are explained in “Gel filtration: Principles and Methods.” 8th ed., Amersham Pharmacia Biotech AB, Rahhms I Lund, Uppsala, Sweden. The appropriate columns for fractionating particular ranges can be readily selected and effectively used to resolve the desired fractions, e.g., SEPHACRYL™ S 100 HR, SEPHACRYL™ S 200 HR, SEPHACRYL™ S 300 HR, SEPHACRYL™ S 400 HR and SEPHACRYL™ S 500 HR or their equivalents. In an analogous fashion, SEPHAROSE™ media or their equivalents, e.g., SEPHAROSE™ 6B, 4B, 2B, SEPHADEX™ G-100, can be used. Such columns and column compositions are availably from commercial sources (e.g., Pharmacia in Uppsala, Sweden).

Anion-exchange chromatography can also be used in accordance with the methods and compositions disclosed herein. Anion-exchange chromatography, for example, can be used to further separate Chlorella extract, components of Chlorella extract, fractions and sub-fractions of Chlorella extract, precipitates of Chlorella extract, etc. Anion-exchange chromatography can be accomplished by any method known in the art, such as those described in “Ion exchange chromatography,” by James S. Fritz and Douglas T. Gjerde (Weinheim; New York: Wiley-VCH, 2000). It should be appreciated that large molecular weight species present in Chlorella extract (e.g., high molecular weight carbohydrates) can be readily separated from bulk compositions using anion-exchange chromatography.

Purification and/or separation of a component retrieved from a Chlorella (e.g., a polysaccharide, polysaccharide complex including a polysaccharide complex with protein) extract can also be achieved using other chromatography techniques, including affinity chromatography, ion-exchange chromatography, hydrophobic interaction chromatography, etc. Ultrafiltration of Chlorella extract components can also be performed using molecular membranes with appropriate molecular mass cut-offs. The specific membranes and procedures used to effect fractionation are available to those skilled in the art.

Ultrafiltration of the samples can be performed using molecular membranes with appropriate molecular mass cut-offs. The specific membranes and procedures used to effect fractionation are widely available to those skilled in the art.

In one example, a method used for characterizing and quantifying Chlorella extract materials can be based on combined size exclusion chromatography (SEC)/multi-angle laser light scattering (MALS)/refractive index detection (RI). In the hybrid technique (SEC/MALS/RI), an isocratic HPLC experiment using a Tosohaas GMPWXL SEC column can be used to separate mixtures according to molecular size. On-line MALS can determine the average molecular weight distribution of eluting components and hence provides specificity in the analysis. RI detection can be used both for quantification and to provide the elution profile required in processing the MALS data.

It is understood that the fractionation methods disclosed herein can be used in combination with the various compositions disclosed herein, methods disclosed herein, products disclosed herein, and applications of the herein disclosed subject matter. It is also understood that any composition obtained from the methods disclosed herein are contemplated for with the methods and applications disclosed herein.

### Characterization of Fractions

Carbohydrate composition, nucleic acid (e.g., DNA content) and amino acid composition of the Chlorella extracts can be determined by any suitable method known in the art.

Immune activity of the disclosed extracts can be associated with Chlorella polysaccharides, defined as those macromolecules consisting of monosaccharides joined by glycosidic linkages. The polysaccharides can be present in the extracts in the form of free polysaccharides or complexed polysaccharides (i.e. polysaccharides which are non-covalently associated with a non-polysaccharide biopolymer which, by itself, has no significant immune activity). In one aspect, the protein content of the extract can be about 20% to 50%, or 20% to 30%. Of this percentage of proteins, about 40% to 60% can be associated with polysaccharides.

Non-polysaccharide biopolymers include nucleic acid polymers (e.g., DNA), protein and RNA, which can contribute to the cumulative molecular weight of the extract but which has no significant immune activity. Unassociated RNA, DNA and protein, i.e. those not complexed with the polysaccharides, do not necessarily contribute significantly to immune activity of the extracts. For the purposes of the present application, unassociated RNA,

DNA and protein are defined functionally as those RNA, DNA and protein which are susceptible to cleavage by ribonuclease (RNAse), deoxyribonuclease (DNAse) and common proteases of the serine and thiol class. The extracts disclosed herein can thus be essentially free or substantially free of unassociated RNA, DNA and protein. By “essentially free” is meant less than 5% unassociated DNA or RNA and less than 15% unassociated proteins. By “substantially free” is meant less than 2% associated DNA or RNA and less than 10% unassociated proteins.

While non-polysaccharide biopolymers per se lack immune activity, their association with the polysaccharides can contribute to the immune activity of the polysaccharides since the non-polysaccharide biopolymers of the complex can fulfill certain steric or polar requirements which enable the polysaccharides to function effectively as immunomodulators.

A fraction or fractions obtained from any of the aforementioned methods or other methods in accordance with the disclosed subject matter can be characterized to elucidate appropriate physical and chemical properties of the fraction(s). Physical and chemical characterization methods can be used on modified or unmodified fractions obtained through the practice of the methods disclosed herein.

Physical and chemical properties (including structural information) can be obtained by any method known in the art. Examples include the use of solution and solid-state nuclear magnetic resonance (NMR) spectroscopy, infrared spectroscopy (IR), mass spectrometry (MS), and UV-vis spectroscopy. Specifically, ¹H, ¹³C, and ³¹P NMR can be used to ascertain chemical and structural properties of the fractions obtained from the methods disclosed herein. A variety of 1-D and 2-D NMR methods can be used (on any appropriate nucleus), including Distortionless Enhancement by Polarization Transfer (DEPT and DEPTQ for quaternary nuclei), Heteronuclear Single Quantum Coherence (HSQC), Heteronuclear Multiple Bond Correlation (HMBC), Correlation Spectroscopy (COSY), Totally Correlated Spectroscopy (TOCSY), and Nuclear Overhauser Effect (NOE) difference spectroscopy, among others. These methods and other methods are described in more detail in “Spectrometric Identification of Organic Compounds,” 7^{th} ed., by Robert M. Silverstein and Francis X. Webster and David J. Kiemle (Wiley & Sons: New York, 2005).

Chemical modifications of the fractions disclosed herein can be carried out to determine the presence and/or absence of functional groups and thereby further elucidate structural information. Dephosphorylation can be used to remove phosphorylated functional groups from a fraction. Dephosphorylation can be carried out, for example, enzymatically or chemically (e.g., with HF). Deacetylations (e.g., de-O-acetylation) can be carried out using an appropriate base (e.g., NH₄OH).

### Fraction Compositions

Fraction compositions obtained from the methods disclosed herein can comprise a polysaccharide and/or polysaccharide complexes. By “polysaccharide complex,” it is meant that one or more polysaccharides are non-covalently associated with a non-polysaccharide biopolymer. Examples of non-polysaccharide biopolymers that the herein disclosed polysaccharides can associate with comprise, inter alia, nucleic acids as described herein before (e.g., DNA, RNA) and proteins. It should be appreciated that such non-covalently associated non-polysaccharide biopolymers can contribute to the cumulative molecular weight of the polysaccharide, but such biopolymers are generally thought to have little to no impact on the immunomodulating properties of the polysaccharides.

In another aspect, the polysaccharide and polysaccharide complexes can be substantially free of ribose, nucleic acids, ribonucleic acids and unassociated protein. The polysaccharide and polysaccharide complexes can also optionally contain N-acetyl glucosamine and N-acetyl galactosamine.

In yet another aspect, the disclosed extracts retain immunomodulating activity upon treatment to remove unassociated nucleic acids (e.g., DNA, RNA) and proteins. Such treatment includes digestion by pronase, DNAse, RNAse and proteases.

In another aspect, the disclosed extracts can retain immunomodulating activity upon treatment to effect cleavage of specific glycosidic linkages, the linkages being defined by their susceptibility to cleavage by amylase, amyloglucosidase, cellulase or neuraminidase. Such susceptible linkages typically comprise: (i) three or more α-1,4-linked D-glucose units; (ii) α-1,4-linked glucosides; (iii) α-1,4-linked galactosides; or (iv) α-1,4-linked D-glucose.

A polysaccharide or polysaccharide complex obtained from Chlorella can have a molecular weight of from about 1×10³ to about 1×10⁶ Da, or from about 1×10³ to about 1×10⁵ Da. In one embodiment, the disclosed polysaccharides or polysaccharide complexes can have a molecular weight of from about 1×10³ to about 3×10³ Da. In another embodiment, the disclosed polysaccharides or polysaccharide complexes can have a molecular weight from about 2×10³ to about 4×10³ Da. In a further embodiment, the disclosed polysaccharides or polysaccharide complexes can have a molecular weight from about 3×10³ to about 5×10³ Da. In a still further embodiment, the disclosed polysaccharides or polysaccharide complexes can have a molecular weight from about 4×10³ to about 6×10³ Da. In a yet still further embodiment, the disclosed polysaccharides or polysaccharide complexes can have a molecular weight from about 5×10³ to about 7×10³ Da. In a yet still further embodiment, the disclosed polysaccharides or polysaccharide complexes can have a molecular weight from about 6×10³ to about 8×10³ Da. In a yet another embodiment, the disclosed polysaccharides or polysaccharide complexes can have a molecular weight from about 7×10³ to about 9×10³ Da. In a still another embodiment, the disclosed polysaccharides or polysaccharide complexes can have a molecular weight from about 8×10³ to about 1×10⁴ Da. In another further embodiment, the disclosed polysaccharides or polysaccharide complexes can have a molecular weight from about 9×10³ to about 2×10⁴ Da. In a yet another further embodiment, the disclosed polysaccharides or polysaccharide complexes can have a molecular weight from about 1×10⁴ to about 3×10⁴ Da. In a still yet another further embodiment, the disclosed polysaccharides or polysaccharide complexes can have a molecular weight from about 2×10⁴ to about 4×10⁴ Da. In one further embodiment, the disclosed polysaccharides or polysaccharide complexes can have a molecular weight from about 3×10⁴ to about 5×10⁴ Da. In one yet further embodiment, the disclosed polysaccharides or polysaccharide complexes can have a molecular weight from about 4×10⁴ to about 6×10⁴ Da. In another embodiment, the disclosed polysaccharides or polysaccharide complexes can have a molecular weight from about 5×10⁴ to about 7×10⁴ Da. In one further embodiment, the disclosed polysaccharides or polysaccharide complexes can have a molecular weight from about 6×10⁴ to about 8×10⁴ Da. In a yet still further embodiment, the disclosed polysaccharides or polysaccharide complexes can have a molecular weight from about 7×10⁴ to about 9×10⁴ Da. In a yet another embodiment, the disclosed polysaccharides or polysaccharide complexes can have a molecular weight or from about 8×10⁴ to about 1×10⁵ Da. Non-limiting examples of polysaccharide or polysaccharide complexes can have a molecular weight of about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, or about 1×10⁵ Da. It is understood, however, that the disclosed polysaccharides or polysaccharide complexes can have any molecular weight from about 1×10³ to about 1×10⁶ Da.

Monosaccharide residues that can be present in the disclosed polysaccharides and polysaccharide complexes comprise, without limitation, mannose, rhamnose, glucose, galactose, arabinose, and any combination thereof. Contemplated polysaccharides comprise monosaccharide residues that exist in D form, in pyranose and/or furanose form. Further, monosaccharides can exist in α and/or β anomeric forms. For example, α-D-mannose and/or β-D-galactose can be present in a polysaccharide. Monosaccharides can be linked together through any appropriate bond sites. Examples comprise monosaccharides linked together through 1→6, 1→4, and 1→3 bonds. Monosaccharide residues can be O-methylated, O-acetylated, O-phosphorylated, and any combination thereof. In one example, a polysaccharide comprises at least two terminal monosaccharides linked to the polysaccharide backbone through phosphodiester bond. By “terminal” is meant at the end of a branch in a branched polysaccharide backbone. A phosphodiester bond can link at least two monosaccharides together through a 1→6 bond in a polysaccharide, for example, through a 1-HPO₃→6 bond.

Other examples comprise polysaccharides and polysaccharide complexes comprising a glucose-side chain attached to (i.e., bonded to) every second galactose. A polysaccharide or polysaccharide complex can also comprise two single glucoses attached to adjacent galactoses.

Ratios between individual residues can exist in the present polysaccharides and polysaccharide complexes. An example comprises a polysaccharide wherein the ratio of galactose to glucose is about 2:1. Another example comprises a polysaccharide wherein the ratio of galactose to glucose is about 3:1. Other contemplated ratios of galactose to glucose comprise, without limitation, about 1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1. Further examples comprise ratios of galactose to glucose including, without limitation, about 1:1.2, 1.3:1, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3. A specific example comprises a polysaccharide wherein the ratio of galactose to glucose is about 2:1, as determined by NMR spectroscopy. Another example comprises a polysaccharide wherein the ratio of galactose to glucose is about 3:1, according to analysis with alditol acetates.

Polysaccharides having phosphosaccharide structures (termed phosphoglycans) have been found to occur in nature in the capsules of bacteria, the cell walls of bacteria and yeast, as well as in the extracellular and cell-surface glycopolymers of Leishmania protozoan parasites, and in glycan chains of some animal glycoproteins.

Capsular polysaccharides (primary serotype-specific antigens in many bacteria) and O-specific polysaccharide chains (somatic antigen) from the cell wall LPS of Gram-negative bacteria usually contain regular poly(glycosyl phosphate) structures with highly diverse monosaccharide representations that have been reviewed elsewhere. Amongst the glycosyl phosphate units described, α-D-GlcpNAc 1-phosphate, α-D-Glcp 1-phosphate, α-D-GalpNAc 1-phosphate, α-D-Galp 1-phosphate and α-L-Rhap 1-phosphate are the most widely distributed. The cell walls of Gram-positive bacteria also contain anionic glycopolymers that are known to contain repeating aldetol-phosphate units called teichoic acids. The latter group includes poly(glycerol phosphates), poly(erythritol phosphates), poly(ribitol phosphates), poly(arabinitol phosphates) and poly(mannitol phosphates) with a phosphodiester linkage occurring mainly between primary hydroxyl groups, while secondary hydroxyls are unsubstituted or glycosylated. None of the teichoic acid-like polymers contain glycosyl phosphate units.

Unlike bacterial phosphoglycans containing glycosyl phosphate residues, where it is rarely found, α-D-Manp 1-phosphate is commonly found in the phosphoglycans of yeasts. The only known report of glycosyl phosphate structure containing α-Manp 1-phosphate from bacteria is a cell-surface phosphomannan from the Gram-negative bacterium P. gingivalis. The polysaccharide consists of a tri-saccharide repeating unit of →6)-α-D-Manp residues with O-2 side chains of α-D-Manp, α-D-Manp-(1→2)-α-D-Manp and α-D-Manp-(1→2)-α-D-Manp-(1-PO₃H→.

Phosphomannans in yeasts can either form an intracellular slime providing adhesive properties for yeast cells or are part of the cell wall, where they often determine the antigenic specificity of the cells. In contrast to bacterial phosphoglycans, yeast phosphomannans are rarely regular and consist mostly of a backbone of α-(1→6) linked mannopyranosyl units with side chains of various lengths with α-(1→2), α-(1→3) and, sometimes, β-(1→2) glycosidic linkages. The majority of the yeasts phosphomannans possess an α-D-Manp-(1-PO₃H→6)-α-D-Manp-(1→ glycosyl phosphodisaccharide unit in the side chains.

Hydrophilic and hydrophobic phosphoglycans have been shown to comprise culture supernatants and the cell-surface, respectively, of Leishmania promastigotes, a genus of sandfly transmitted protozoan parasites that cause a variety of debilitating and often fatal diseases in humans. Hydrophilic phosphoglycans contain a poly(glycosyl phosphate) structure consisting of linear and ramified (depending on the species) galactomannosyl phosphate repeating units, whereas in hydrophobic phosphoglycans the corresponding poly(glycosyl phosphate) is attached at the reducing end of the chain to a glycan core linked to an inositolphospholipid anchor to make a lipophosphoglycan conjugate. The sequence α-D-Manp-(1-PO₃H→6)-β-D-Galp-(1→ is the most frequently found glycosyl phosphosaccharide unit in phosphoglycans of Leishmania parasites.

Glycosyl phosphosaccharide structures have been also been found to comprise glycoproteins of animal origin. The sequence α-D-Glcp-(1-PO₃H→6)-α-D-Manp-(1→ has been found to be a terminal fragment in the high-mannose type oligosaccharide chains of some plasma membrane and cytoplasmatic recognition glycoproteins, whereas the sequence α-D-GlcpNAc-(1-PO₃H→6)-α-D-Manp-(1→ has been found to be a component of a number of lysosomal enzymes.

The majority of glycosyl phosphate units found in natural phosphoglycans of diverse origin have been found to have an α-D- or α-L-hexopyranose configuration, with the phosphate group occupying an axial position at C-1, which is known to be favored by the anomeric effect.

Polysaccharides from microalgae have not been studied in as much detail as those from bacteria, yeasts and plants. Disclosed herein are phosphoglycans from microalgae origin containing a glycosyl phosphate structure. Side chains of α-D-Manp-(1-PO₃H→ units is a structural feature that resemble some yeast phosphoglycans structures. However, no reports have been made so far concerning the presence of methylated phosphosaccharide units, 3-O-methyl α-Manp-(1-PO₃H→ in this case, which makes the structure reported herein unique.

The cell wall of the Gram-negative bacteria Spirillaplanes (Micromonospora) yamanashiensis was found to contain an anionic polymer consisting of repeating →6)-α-D-GlcpNAc-(1→6)-α-D-GlcpNAc-(1-PO₃H→ units with 3-O-methyl-α-mannopyranosyl residues at position 3 (50%) of some 6-phosphorylated N-aceylglucosamine residues. This side chain of 3-O-methyl-α-Manp however, did not appear to be phosphorylated.

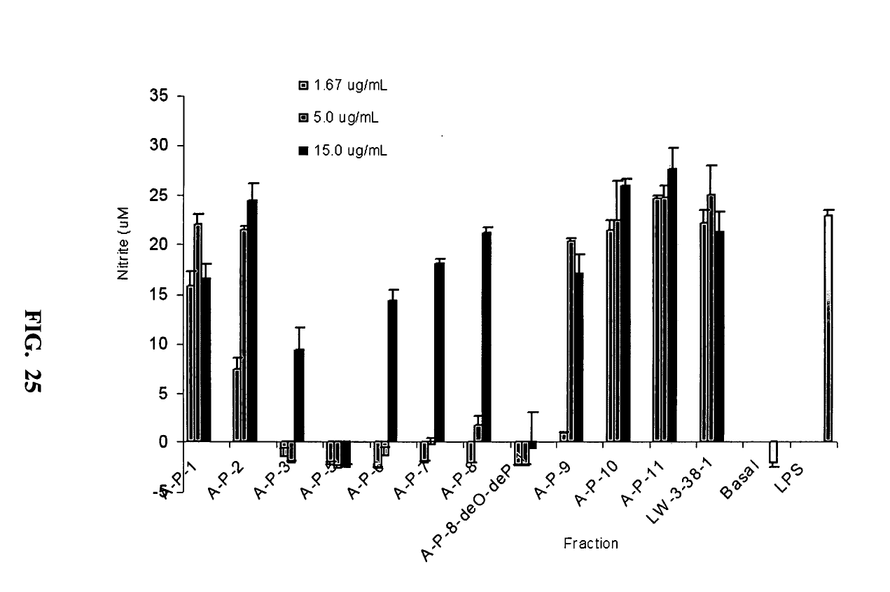

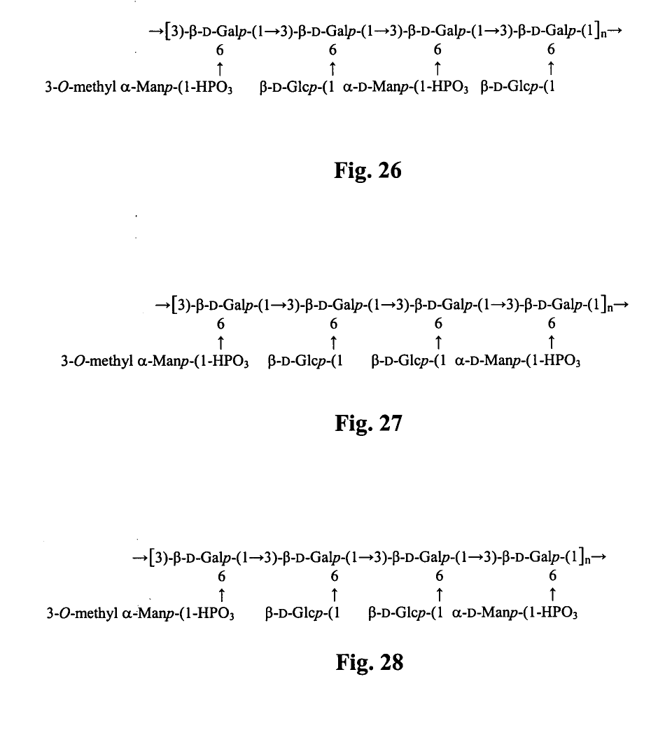

The following are non-limiting examples of the disclosed polysaccharides presented according to their IUPAC designation. These formulae are also referenced in FIGS. 26-34.

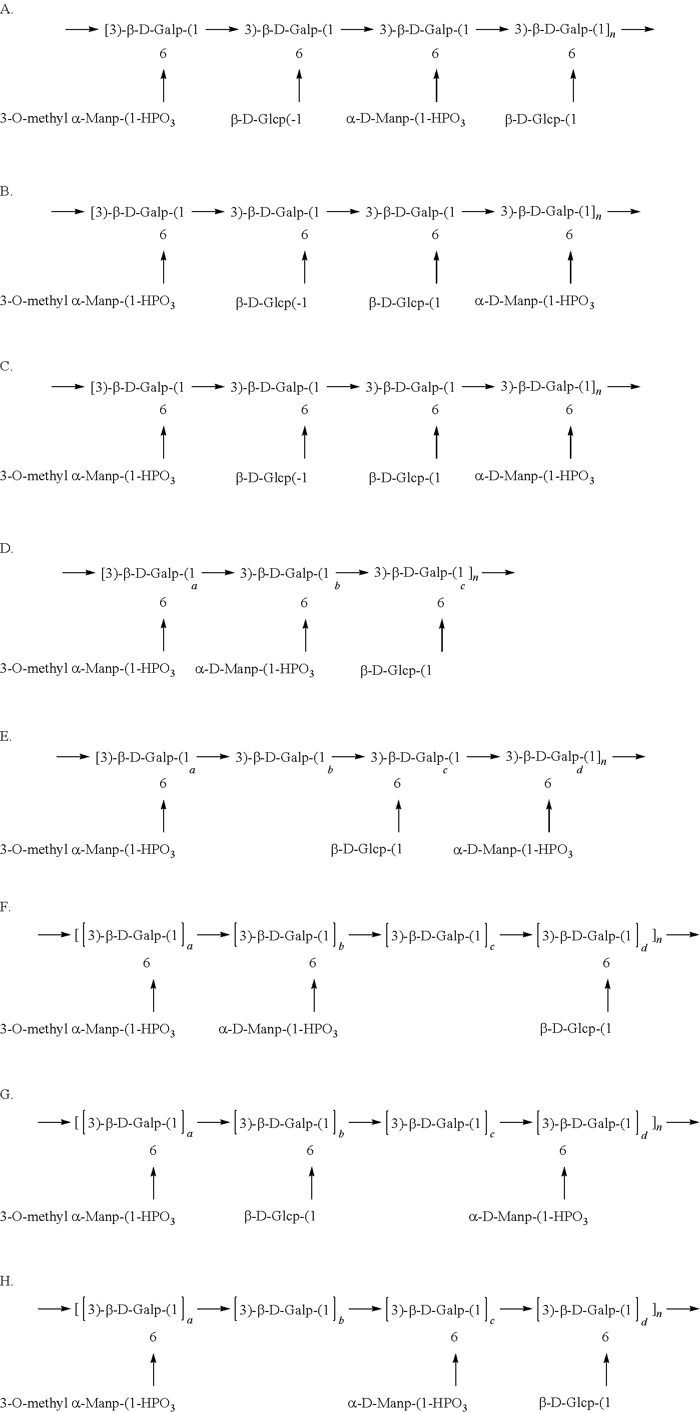

wherein the indices a+b+c+d=n. The index n reflects the average molecular weight of the disclosed polysaccharide or polysaccharide complex as defined herein above. As such, the index n is from about 5 to about 500. In one embodiment, n is from about 7 to about 400. The indices a, b, c, and d can have any value from about 1 to about 450. In one embodiment, the index a is from about 10 to about 50. In a further embodiment, the index a is from about 20 to about 70. In another embodiment, the index a is from about 30 to about 50. In a yet further embodiment the index a is from about 5 to about 15. In one embodiment, the index b is from about 10 to about 50. In a further embodiment, the index b is from about 20 to about 70. In another embodiment, the index b is from about 30 to about 50. In a yet further embodiment the index b is from about 5 to about 15. In one embodiment, the index c is from about 10 to about 50. In a further embodiment, the index c is from about 20 to about 70. In another embodiment, the index c is from about 30 to about 50. In a yet further embodiment the index c is from about 5 to about 15. In one embodiment, the index d is from about 10 to about 50. In a further embodiment, the index d is from about 20 to about 70. In another embodiment, the index d is from about 30 to about 50. In a yet further embodiment the index d is from about 5 to about 15. The indices a, b, c, and d, however, can have any value from 5 to 500.

### Immunomodulating Properties of Disclosed Compositions

While not wishing to be bound by theory, the compositions and compounds disclosed herein can be biological response modifiers (immunostimulants or immunomodulators). Biological response modifiers are defined as those agents that modify the subject's subject's biological response by a stimulation of the immune system, which can result in various therapeutic effects. One of the categories of substances belonging to this class is immunomodulators. As such, the disclosed compositions can be used to modulate an immune response. In the context of the disclosed subject matter, such modulation can be an enhancement of the subject's immunity defense mechanism.

Chlorella extracts are B-cell and macrophage stimulators. One benefit of B-cell immunomodulators is that they can stimulate immune function in subjects who have an impaired antibody response to an antigen. Also, a B-cell stimulator can increase the efficacy of the antibody immune response when presented with a new infection. Chlorella extracts provide a safe, efficacious and cost effective alternative for preventative health treatment.

Disclosed herein are in vitro studies that demonstrate that Chlorella extracts stimulate proliferation of BALB/c mouse spleen cells, and macrophage production of IL-6 and NO₂. Further disclosed herein are in vivo studies that indicate that Chlorella extracts can significantly reduce infection with Listeria monocytogenes, as well as the fungus Candida albicans.

The immunostimulatory activity results of the compositions disclosed herein based on NO production by peritoneal macrophages, are shown in FIG. 25. Two fractions obtained through size exclusion chromatography, referred to hereinafter as “A-P-1” and “A-P-2” were active as immunostimulants. It should be appreciated that it can be noticeable that the immunostimulatory activity of the fractions derived from anion exchange chromatography of fraction A-P-1 increases with the molecular size, which is an indication that for this phosphorylated polysaccharide to be active as immunostimulant, a minimum number of repeating units can be preferred. The graph also shows that the polysaccharide completely loses its immunostimulatory effect after removal of both the acetyl and phosphate groups (fraction A-P-8-deO-deP).

A series of three toxicology trials were completed for Chlorella extracts. No effect of Chlorella extract administration was evident during a 28-day oral toxicity study in rats. For the acute oral toxicity in rats, to determine the highest non-lethal or the lowest lethal dose of the product following a single oral administration, the study found that the lowest lethal dose of a crude Chlorella extract was in excess of 2000 mg/kg body weight. The bacterial mutation assay showed that Chlorella extracts did not exhibit any mutagenic activity under the test conditions.

A randomized, double-blind placebo-controlled study was conducted and indicated that Chlorella extracts demonstrated significant immunostimulatory effects in healthy adults receiving the influenza vaccine, compared to placebo subjects (see U.S. Pat. No. 6,551,596). In vitro experiments with human blood cells show stimulation of production of interleukins, similar to that seen in the mouse model.

### Compositions

Disclosed herein are compositions comprising:
a) one or more polysaccharide or polysaccharide complexes, comprising:
   i) at least one methylated phosphosaccharide unit having the formula:

3-O-methyl α-Manp-(1-PO₃H→; or
ii) at least one phoshosaccaride unit having the formula:

α-D-Manp-(1-PO₃H→;
wherein the polysaccharide or polysaccharide complex has a molecular weight of from about 1×10³ to about 1×10⁶ Da; and
b) one or more adjunct ingredients.

As such, in one embodiment the compositions can comprise:
a) one or more polysaccharide or polysaccharide complexes, comprising at least one methylated phosphosaccharide unit having the formula:

3-O-methyl α-Manp-(1-PO₃H→; or
wherein the polysaccharide or polysaccharide complex has a molecular weight of from about 1×10³ to about 1×10⁶ Da; and
b) one or more adjunct ingredients.

In a further embodiment, the compositions can comprise:
a) one or more polysaccharide or polysaccharide complexes, comprising at least one phoshosaccaride unit having the formula:

α-D-Manp-(1-PO₃H→;
wherein the polysaccharide or polysaccharide complex has a molecular weight of from about 1×10³ to about 1×10⁶ Da; and
b) one or more adjunct ingredients.

In another embodiment, the compositions can comprise:
a) one or more polysaccharide or polysaccharide complexes, comprising:
   i) at least one methylated phosphosaccharide unit having the formula:

3-O-methyl α-Manp-(1-PO₃H→; and
ii) at least one phoshosaccaride unit having the formula:

α-D-Manp-(1-PO₃H→;
wherein the polysaccharide or polysaccharide complex has a molecular weight of from about 1×10³ to about 1×10⁶ Da; and
b) one or more adjunct ingredients.

As used herein “adjunct ingredient” means another ingredient which can be pharmacologically active or which can be inert. For example, inert ingredients can be liquid carriers, solid excipients, stabilizers, surfactants, and the like. Pharmacologically active adjuncts can be analgesics, opiods, immunosuppressants, antibacterial agents, and the like.

### Pharmaceutical Compositions

Disclosed herein are pharmaceutical composition comprising:
a) one or more polysaccharide or polysaccharide complexes, comprising:
   i) at least one methylated phosphosaccharide unit having the formula:

3-O-methyl α-Manp-(1-PO₃H→; or
ii) at least one phosphosaccaride unit having the formula:

α-D-Manp-(1-PO₃H→;
wherein the polysaccharide or polysaccharide complex has a molecular weight of from about 1×10³ to about 1×10⁶ Da; and
b) one or more pharmaceutically acceptable ingredients.

As such, in one embodiment the compositions can comprise:
a) one or more polysaccharide or polysaccharide complexes, comprising at least one methylated phosphosaccharide unit having the formula:

3-O-methyl α-Manp-(1-PO₃H→; or
wherein the polysaccharide or polysaccharide complex has a molecular weight of from about 1×10³ to about 1×10⁶ Da; and
b) one or more pharmaceutically acceptable ingredients.

In a further embodiment, the compositions can comprise:
a) one or more polysaccharide or polysaccharide complexes, comprising at least one phoshosaccaride unit having the formula:

α-D-Manp-(1-PO₃H→;
wherein the polysaccharide or polysaccharide complex has a molecular weight of from about 1×10³ to about 1×10⁶ Da; and
b) one or more pharmaceutically acceptable ingredients.

In another embodiment, the compositions can comprise:
a) one or more polysaccharide or polysaccharide complexes, comprising:
   i) at least one methylated phosphosaccharide unit having the formula:

3-O-methyl α-Manp-(1-PO₃H→; and
ii) at least one phoshosaccaride unit having the formula:

α-D-Manp-(1-PO₃H→;
wherein the polysaccharide or polysaccharide complex has a molecular weight of from about 1×10³ to about 1×10⁶ Da; and
b) one or more pharmaceutically acceptable ingredients.

For the purposes of the present disclosure the term “excipient” and “carrier” are used interchangeably throughout the description of the present disclosure and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present disclosure have improved cellular potency, pharmacokinetic properties, as well as improved oral bioavailability.

Chlorella extracts and fractions disclosed herein can be suitable for use in any condition or disease state where immune response modulation is desired. In one example, the disclosed compositions can be used in an effective amount as adjuvants in various forms of mucosal vaccine preparations, e.g., for oral administration.

As used herein the term “adjuvant” means a pharmaceutically acceptable ingredient, for example, pharmacological or immunological agents that modify the effect of other agents (e.g., drugs, vaccines, immunosuppressants, or biologically active agents) while having few if any direct effects when given by themselves.

Adjuvants can protect the antigen from rapid dispersal by sequestering it in a local deposit, or they can contain substances that stimulate the subject to secrete factors that are chemotactic for macrophages and other components of the immune system. Known adjuvants for mucosal administration include bacterial toxins, e.g., the cholera toxin (CT), the E. coli heat-labile toxin (LT), the Clostridium difficile toxin A and the pertussis toxin (PT). Chlorella extracts and fractions, being an edible product of high molecular weight and themselves immune stimulants, are candidates for use as adjuvants in oral vaccines.

The present disclosure also provides a method for modulating the immune response of a subject (e.g., a mammal including a human) by administering to the subject an effective amount of a composition disclosed herein. Such modulation includes increased proliferation of splenocytes and increased production of cytokines such as IL-6, IL-10, INF-γ and TNF-α, and can be advantageously used to treat or prevent bacterial or fungal infections.

The extract can further be administered as a supplement to a vaccination regimen to further stimulate the immune response. A flu vaccine, for example, can be advantageously used with the extract. The extract can be present as an adjuvant to the vaccines, especially as an oral vaccine adjuvant.

A suitable pharmaceutical composition can comprise any of the disclosed polysaccharide or polysaccharide complexes and other bioactive agents, along with a pharmaceutically acceptable ingredient, for example, a pharmaceutically acceptable carrier. In some examples, the compositions disclosed herein can themselves be pharmaceutically acceptable carriers. The pharmaceutical formulations disclosed herein can be used therapeutically or prophylactically.

By “pharmaceutically acceptable carrier” is meant a material that is not biologically or otherwise undesirable, i.e., the material can be administered to a subject without causing any undesirable biological effects or interacting in a deleterious manner with any of the other components of the pharmaceutical formulation in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. Suitable carriers and their formulations are described in Remington: The Science and Practice of Pharmacy, 21 st Ed., Lippincott Williams & Wilkins, Philadelphia, Pa., 2005, which is incorporated by reference herein for its teachings of carriers and pharmaceutical formulations. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution can be from about 5 to about 8 (e.g., from about 7 to about 7.5). Further carriers include sustained release preparations such as semipermeable matrices of solid hydrophobic polymers containing the disclosed compounds, which matrices are in the form of shaped articles, e.g., films, liposomes, microparticles, or microcapsules. It will be apparent to those persons skilled in the art that certain carriers can be more preferable depending upon, for instance, the route of administration and concentration of composition being administered. Other compounds can be administered according to standard procedures used by those skilled in the art.

Pharmaceutical formulations can include additional carriers, as well as thickeners, diluents, buffers, preservatives, surface active agents and the like in addition to the compounds disclosed herein. Pharmaceutical formulations can also include one or more additional active ingredients such as antimicrobial agents, anti-inflammatory agents, anesthetics, and the like.

The pharmaceutical formulation can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, marine oils, and injectable organic esters such as ethyl oleate. Aqueous carriers include water, alcoholic/aqueous solutions, and emulsions or suspensions, including saline and buffered media. Parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Also provided herein are nutritional compositions containing the Chlorella extract with at least one energy source which can be carbohydrates, fats or nitrogen.

### Nutritional Compositions

Disclosed herein are compositions comprising:
a) one or more polysaccharide or polysaccharide complexes, comprising:
   i) at least one methylated phosphosaccharide unit having the formula:

3-O-methyl α-Manp-(1-PO₃H→; or
ii) at least one phoshosaccaride unit having the formula:

α-D-Manp-(1-PO₃H→;
wherein the polysaccharide or polysaccharide complex has a molecular weight of from about 1×10³ to about 1×10⁶ Da; and
b) one or more comestible or nutritional ingredients.

As such, in one embodiment the compositions can comprise:
a) one or more polysaccharide or polysaccharide complexes, comprising at least one methylated phosphosaccharide unit having the formula:

3-O-methyl α-Manp-(1-PO₃H→; or
wherein the polysaccharide or polysaccharide complex has a molecular weight of from about 1×10³ to about 1×10⁶ Da; and
b) one or more comestible or nutritional ingredients.

In a further embodiment, the compositions can comprise:
a) one or more polysaccharide or polysaccharide complexes, comprising at least one phoshosaccaride unit having the formula:

α-D-Manp-(1-PO₃H→;
wherein the polysaccharide or polysaccharide complex has a molecular weight of from about 1×10³ to about 1×10⁶ Da; and
b) one or more adjunct ingredients.

In another embodiment, the compositions can comprise:
a) one or more polysaccharide or polysaccharide complexes, comprising:
   i) at least one methylated phosphosaccharide unit having the formula:

3-O-methyl α-Manp-(1-PO₃H→; and
ii) at least one phoshosaccaride unit having the formula:

α-D-Manp-(1-PO₃H→;
wherein the polysaccharide or polysaccharide complex has a molecular weight of from about 1×10³ to about 1×10⁶ Da; and
b) one or more comestible or nutritional or nutritional ingredients.

What is the term “comestible” means is anything that can be eaten, i.e., food. The disclosed polysaccharide or polysaccharide complex can be combined with any comestible product that is compatible. For example, the disclosed polysaccharide or polysaccharide complex can be added to a beverage, i.e., fruit juices, vegetable juices, colas, and the like. The polysaccharide or polysaccharide complexes can be combined with solid food products, for example, admixed with fruits, yogurt, or with a nutritional supplement.

The nutritional and pharmaceutical compositions comprising the Chlorella extracts and fractions disclosed herein can be formulated and administered in any form suitable for enteral administration, for example oral administration or tube feeding. Nutritional and pharmaceutical formulations can comprise, for example, vitamin E, vitamin C, vitamin B, folic acid, or any combination thereof. Nutritional and pharmaceutical formulations can also comprise, for example, fish oil, fungal oil, algal oil, marine oil, Spirulina, and Echinacea, or any combination thereof. The formulations can be conveniently administered in the form of an aqueous liquid. The formulations suitable for enteral application can be in aqueous form or in powder or granulate form, including tablet form. The powder or granulate can be conveniently added to water prior to use. In liquid form, the compositions can have a solid content of typically from about 0.1% to about 50% by weight. As a drink, the compositions can be obtained by any manner known, e.g., by admixing the Chlorella extract or fraction with an energy source such as a carbohydrate, fat and/or nitrogen source.

The nutritional compositions can be in the form of a complete formula diet (in liquid or powder form), such that when used as the sole nutrition source, essentially all daily caloric, nitrogen, fatty acids, vitamin, mineral and trace element requirements can be met. Nutritional compositions contemplated can comprise, inter alia, polysaccharide and polysaccharide complexes disclosed herein and one or more of a carbohydrate, fat, and/or nitrogen source (e.g., protein).

Pharmaceutical compositions disclosed herein can also be formulated in a single-dose or multi-dose format, where they comprise Chlorella extracts and a pharmaceutically acceptable carrier. Such pharmaceutical compositions can be suitable for enteral administration, such as oral, nasal or rectal administration. Pharmaceutical formulations for oral administration include, but are not limited to, powders or granules, suspensions or solutions in water or non-aqueous media, capsules, gel-caps, sachets, or tablets. Thickeners, flavorings, diluents, emulsifiers, dispersing aids, or binders can be desirable.

Suitable compositions can be in liquid form or solid form. Dosage of liquid compositions can typically range from about 0.1% to about 50% by weight, or from about 1% to about 10% by weight of Chlorella extract or fraction. Dosage of solid compositions can typically range from about 0.2 mg/kg to about 200 mg/kg. The compositions can also be in the form of tablets, hard and soft capsules, and sachets. Suitable carriers are known in the art. They comprise fillers such as sugars or cellulose, binders such as starch, and disintegrators if required or desired.

Pharmaceutical formulations for topical administration can include ointments, lotions, creams, gels, drops, suppositories, sprays, liquids and powders. Conventional pharmaceutical carriers, aqueous, powder or oily bases, thickeners and the like can be desirable.

In another embodiment, the disclosed formulations can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, tartrate, pamoic acid and fumaric acid, or by reaction with an inorganic base such as sodium hydroxide, ammonium hydroxide, potassium hydroxide, and organic bases such as mono-, di-, trialkyl and aryl amines and substituted ethanolamines.

The pharmaceutical compositions may be manufactured using any suitable means, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in a conventional manner using one or more physiologically or pharmaceutically acceptable carriers (vehicles, or diluents) comprising excipients and auxiliaries which facilitate processing of the active compounds into preparations which can be used pharmaceutically. Proper formulation is dependent upon the route of administration chosen.

Any suitable method of administering a pharmaceutical composition to a patient may be used in the methods of treatment of the present disclosure, including injection, transmucosal, oral, inhalation, ocular, rectal, long acting implantation, liposomes, emulsion, or sustained release means.

For injection, the agents of the present disclosure may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. For ocular administration, suspensions in an appropriate saline solution are used as is well known in the art.

For oral administration, the compounds can be formulated readily by combining the active compounds with pharmaceutically acceptable carriers well known in the art. Such carriers enable the compounds of the present disclosure to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl-pyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, such as sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

One type of pharmaceutical carrier for hydrophobic compounds of the present disclosure is a cosolvent system comprising benzyl alcohol, a nonpolar surfactant, a water-miscible organic polymer, and an aqueous phase.

The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may be substituted for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed.

Additionally, the compounds may be delivered using any suitable sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a prolonged period of time. Depending on the chemical nature and the biological stability of the therapeutic reagent, additional strategies for compound stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the agents of the present disclosure may be provided as salts with pharmaceutically acceptable counterions. Salts tend to be more soluble in aqueous or other protic solvents than are the corresponding free base forms.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely exemplary and are not intended to be limiting in scope. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of conditions, e.g., component concentrations, desired solvents, solvent mixtures, temperatures, pressures and other reaction ranges and conditions that can be used to optimize the methods described herein. Only reasonable and routine experimentation will be required to optimize such process conditions.

1. Preparation of Fractionated Crude Chlorella Extract.

Chlorella pyrenoidosa freeze-dried cells (1000 g) were suspended in about 5 L of distillated water and extracted at about 80° C. for about 1 h. After centrifugation (at about 4300 rpm, 30 min, 4° C.), the sediment was re-suspended in distillated water (2.5 L) and extracted under the same conditions. After centrifugation, the supernatants were combined and evaporated in vacuo up to 500 mL to produce the crude extract (CE).

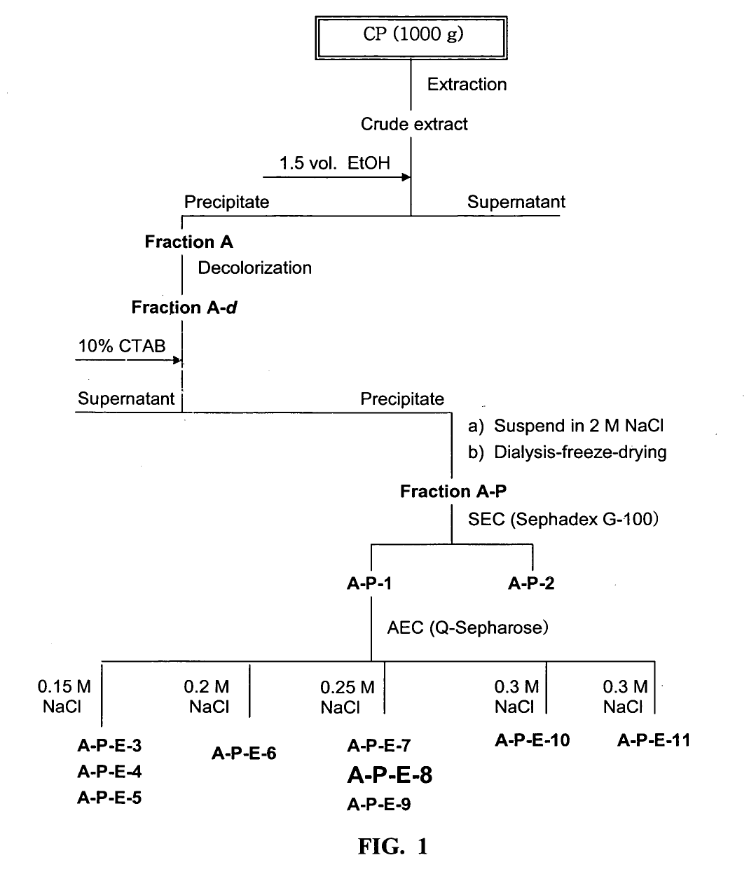

CE was precipitated sequentially with 95% ethanol to produce three precipitates, referred to hereinafter as A, B and C, respectively, after centrifugation, dialysis and freeze-drying. “Precipitate A” was decolorized by stirring with about 2:1 (v/v) CH₃Cl—CH₃OH mixtures (3×500 mL) for 30 min. The resulting “decolorized A” was dissolved in water, dialyzed and freeze-dried to produce fraction “A-d” (FIG. 1).

Fraction A-d was fractionated by treatment with the surfactant cetyltrimethylammonium bromide (CTAB). Aqueous solutions of CTAB [100 mL; 10% (w/v)] and A-d [1 L; 1% (w/v)] were mixed, and the mixture was allowed to stand overnight at about 4° C. After centrifugation, the insoluble portion was dissolved in about 2 L of aqueous NaCl, dialyzed and freeze-dried to yield a fraction herein referred to as “A-P” (FIG. 1).

2. Fractionation of A-P.

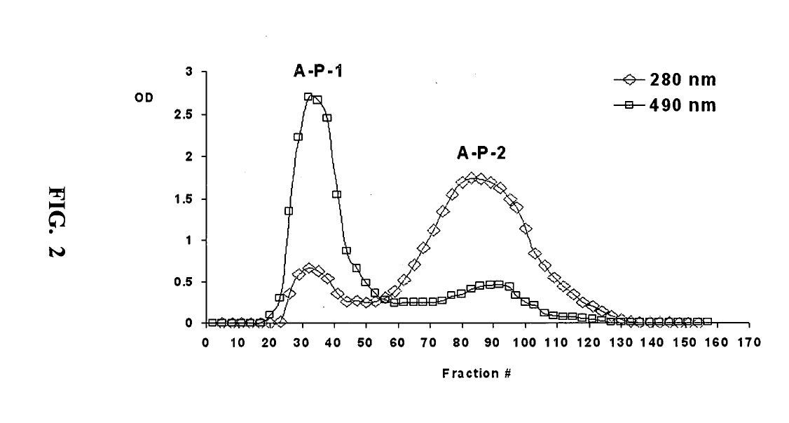

Fraction A-P was separated by size exclusion chromatography on a Sephadex G-100 column (column XK 50/100 Amersham Biosciences, PQ, Canada; 1800 mL bed volume). The sample was dissolved in about 25 mL of about 0.2 M NaCl, filtered through 0.45 μm filters and chromatographed in the same mobile phase at about 1.1 mL/min linear flow rate with collection of 13.2 mL fractions. The separation yielded two fractions (FIG. 2), herein referred to as “A-P-1” (1.8 g) (included for further analysis) and “A-P-2” (250 mg) (excluded from further analysis).

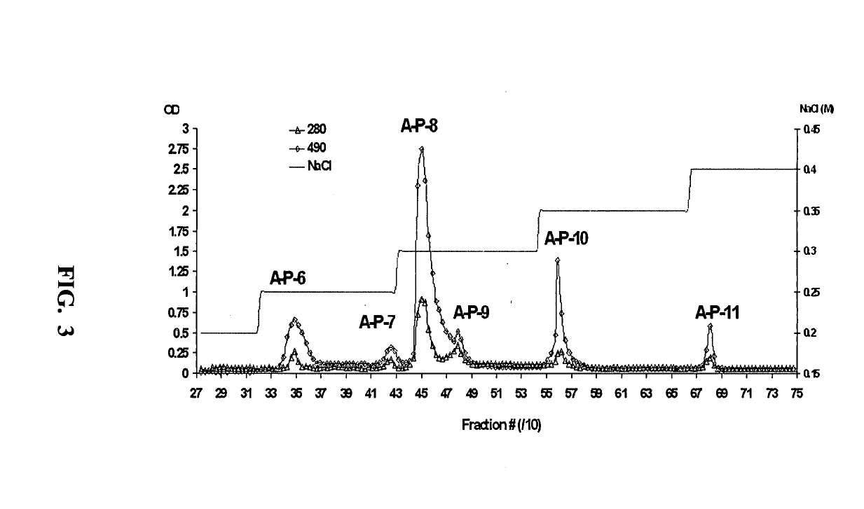

Fraction A-P-1 that contained a higher carbohydrate content was further separated by anion exchange chromatography on a Q-Sepharose Fast Flow column (column XK 26/40, 185 mL bed volume). The sample was dissolved in about 10 mL of distillated water, filtered as mentioned earlier and loaded onto the column at about 1.0 mL/min flow rate. The unbound portion was removed by washing the column off with eight bed volumes of distillated water, and the bound portion was eluted by increasing the ionic strength of the mobile phase in a step-wise fashion, (from zero to 0.4 M NaCl˜ionic strength 0.4), at a flow rate adjusted to about 1.0 mL/min. This separation yielded nine fractions (A-P-3 to A-P-11; the last six (A-P-6 to A-P-11) are shown in FIG. 3. The fraction obtained in the highest yield (A-P-8; 160 mg), which eluted after passing through the column three bed volumes of aqueous 0.3 M NaCl, was chosen for characterization.

3. Characterization of A-P-8.

The ¹³C DEPTQ 135 NMR spectrum of fraction A-P-8 shows major signals having chemical shifts values characteristic of sugars, indicating that a polysaccharide is likely the major component of this fraction. The spectrum also displays signals for a carboxyl group at 173.8 pm and a methyl group at 21.0 ppm, thereby indicating that the polysaccharide was partially O-acetylaed. A sample of the polysaccharide was de-O-acetylated. A 100 mg sample of A-P-8 was treated with 50 mL of 12.5% (v/v) aqueous NH₄OH at about 37° C. for about 16 hours. The de-O-acetylated fraction (A-P-8-deO; 86 mg) was recovered after dialysis and freeze-drying.

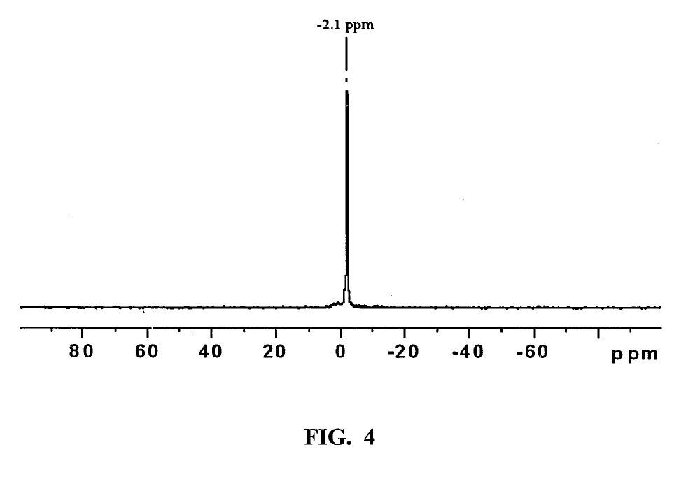

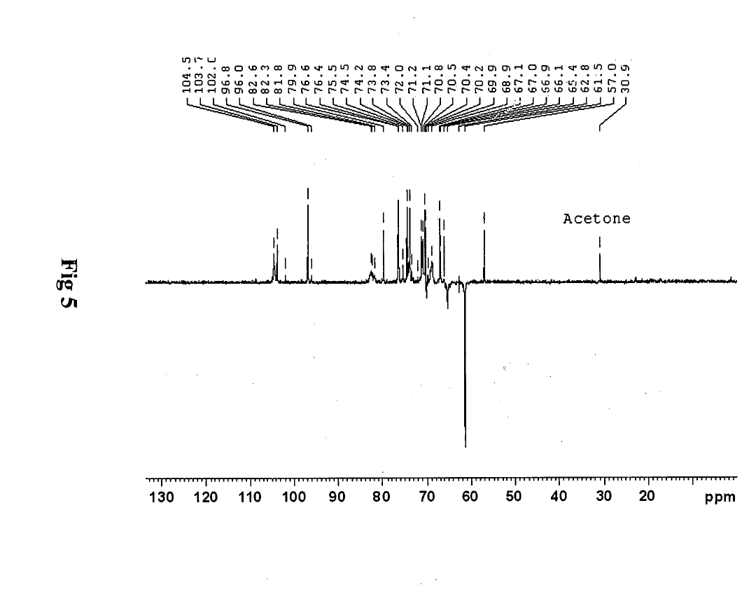

A proton decoupled ³¹P NMR spectrum of fraction A-P-8-deO (FIG. 4) displays a single resonance at approximately −2.1 ppm. This is characteristic of a phosphodiester group, which indicates that the isolated polysaccharide is phosphorylated. A turbidimetric-based analysis of ester sulfate indicates an absence of O-sulfation, while the lack of carboxyl signals in the ¹³C DEPTQ 135 NMR spectrum (FIG. 5) indicates an absence of uronic acids.

The composition of this monosaccharide isolate was determined using a standard alditol acetate method. This method revealed that fraction A-P-8-deO comprises galactose, glucose, mannose and a 3-O-methyl hexose in a 4.5, 1.5, 1.5 to 1 molar ratio, respectively. When this method was implemented following a de-phosphorylation step, the amount of galactose relative to the amounts of glucose, mannose and the 3-O-methyl hexose increased to 10, 1.5, 1.5 to 1.

The polysaccharide of fraction A-P-8-deO (40 mg) was dephosphorylated by treatment with about 3 mL of about a 48% (v/v) aqueous HF solution while kept at about 4° C. for about 48 hours. After removal of the HF by evaporation under a nitrogen stream, the dried mixture was dissolved in water and freeze-dried to yield the de-phosphorylated fraction (A-P-8-deO-deP; 30 mg).

The de-phosphorylated mixture (A-P-deO-deP) was then separated by size exclusion chromatography on a BioGel P-2 (Bio-Rad, Calif., USA) column (XK 16/40; 70 mL bed volume). This sample was dissolved in about 1 mL of deionized water, filtered and chromatographed in the same mobile phase at about 0.5 ml/min flow rate, collecting 0.5 mL fractions.

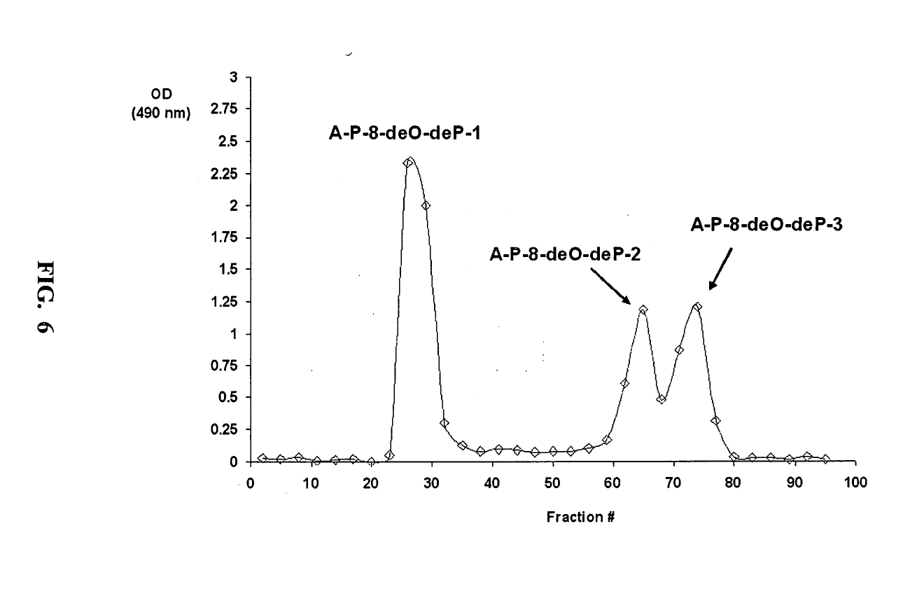

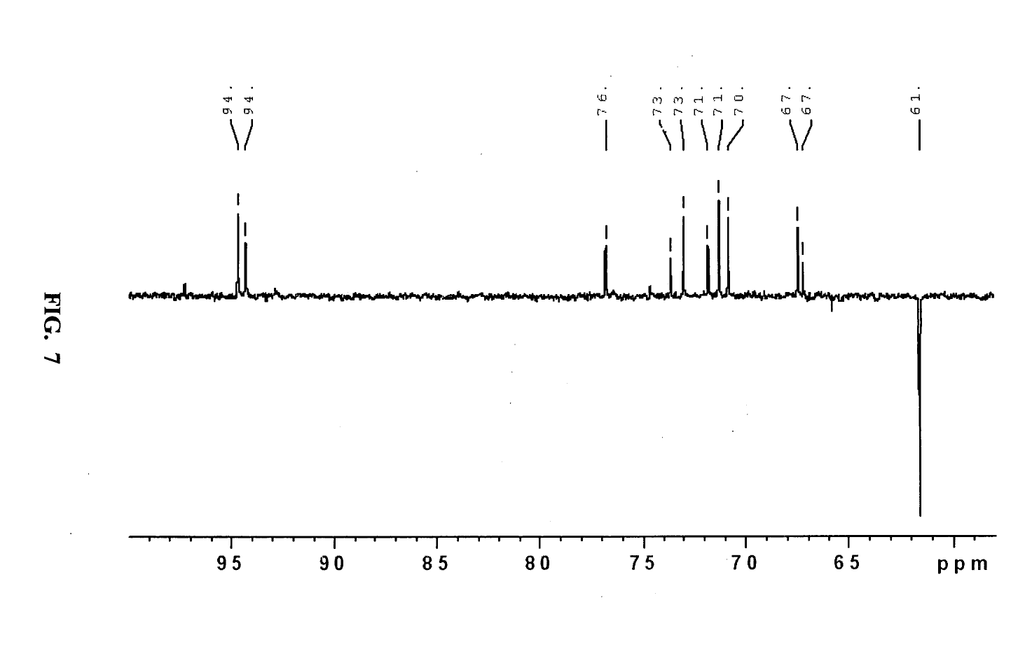

The chromatographic separation yielded three fractions (FIG. 6), a high molecular weight fraction A-P-8-deO-deP-1 (20 mg) that was excluded and used for further analysis and two fractions A-P-8-deO-deP-2 (1.3 mg) and A-P-8-deO-deP-3 (1.4 mg) that were included. These two later fractions eluted with partial overlap, approximately in a 1:1 molar ratio. Analysis of the 1D and the 2D NMR data (¹³C DEPTQ 135 spectra are shown in FIG. 7 and FIG. 8) suggested that fractions A-P-8-deO-deP-3 and A-P-8-deO-deP-2 are comprised of monosaccharide forms of D-mannose and 3-O-methyl-mannose, respectively (chemical shifts are listed in Table 1 and Table 2, respectively).

The data represent two types of structures that are released upon dephosphorylation. In a first type, the two sugars are linked together via a phosphodiester group from the anomeric position of one residue to any position (other than O-1) of the other unit, and the chain is linked to the backbone via a phosphodiester linkage. In the second type, both fragments occur as terminal monosaccharide units (branching units) linked to the polysaccharide backbone, also via a phosphodiester linkage. From the weights of the individual monosaccharides, it was inferred that the ratio of D-manose to 3-O-methyl mannose is 1 to 1.

TABLE 1
¹H NMR and ¹³C NMR chemical shifts (in ppm) of fraction A-P-8-deO-deP-3.

| Monosacch. | H/C-1 | H/C-2 | H/C-3 | H/C-4 | H/C-5 | H/C-6 |
|---|---|---|---|---|---|---|
| α-Manp | 5.22 | 3.97 | 3.88 | 3.69 | 3.85/ | (3.80/3.92)/ |
| | (nd)/94.7 | (nd)/71.3 | (10)/70.9 | (10.4)/67.5 | 73.0 | 61.6 |
| β-Manp | 4.93 | 3.98 | 3.69 | 3.61 | 3.42/ | 3.80/3.92)/ |
| | (nd)/94.3 | (nd)/71.9 | (10)/73.7 | (10.2)/67.3 | 76.8 | 61.6 |

Viccinal ³J_{H, H} coupling constants in Hz appear in brackets.
nd: not detected

TABLE 2
¹H NMR and ¹³C NMR chemical shifts (in ppm) of fraction A-P-8-deO-deP-2.

| Monosacch. | H/C-1 | H/C-2 | H/C-3 | H/C-4 | H/C-5 | H/C-6 | OCH₃ |
|---|---|---|---|---|---|---|---|
| 3-O-methyl | 5.26 | 4.22 | 3.57 | 3.73 | 3.88/ | (3.79/3.92)/ | 3.49/ |
| α-Manp | (nd)/94.7 | (nd)/67.0 | (10)/80.4 | (nd)/66.4 | 73.0 | 61.6 | 56.9 |
| 3-O-methyl | 4.92 | 4.24 | 3.41 | 3.65 | 3.44/ | (3.79/3.92)/ | 3.49/ |
| β-Manp | (nd)/94.4 | (nd)/67.5 | (10)/82.8 | (nd)/66.2 | 76.8 | 61.6 | 56.8 |

Viccinal ³J_{H, H} coupling constants in Hz appear in brackets.
nd: not detected

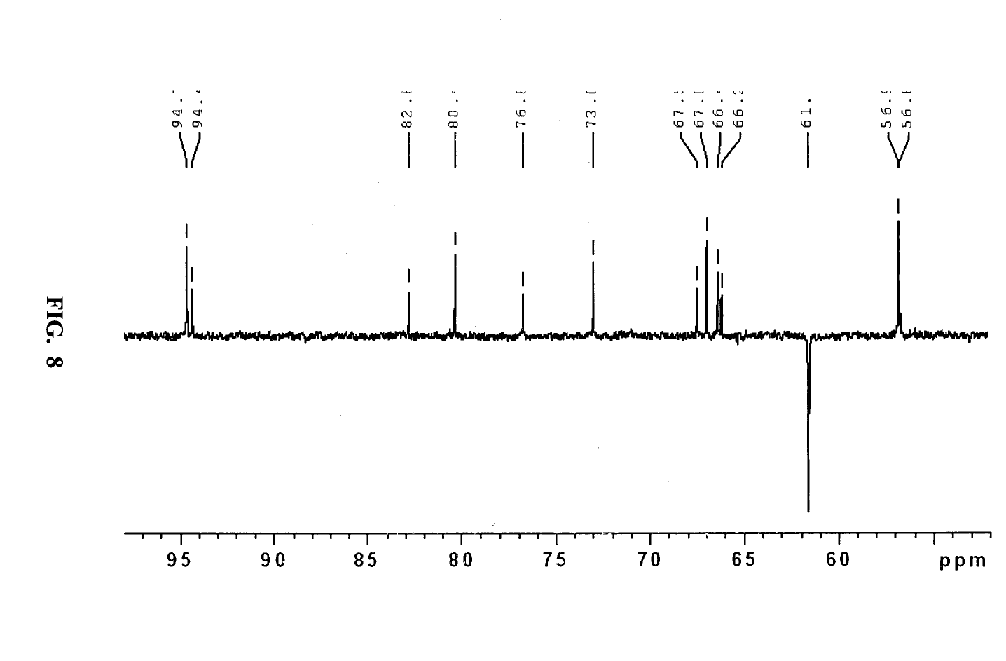

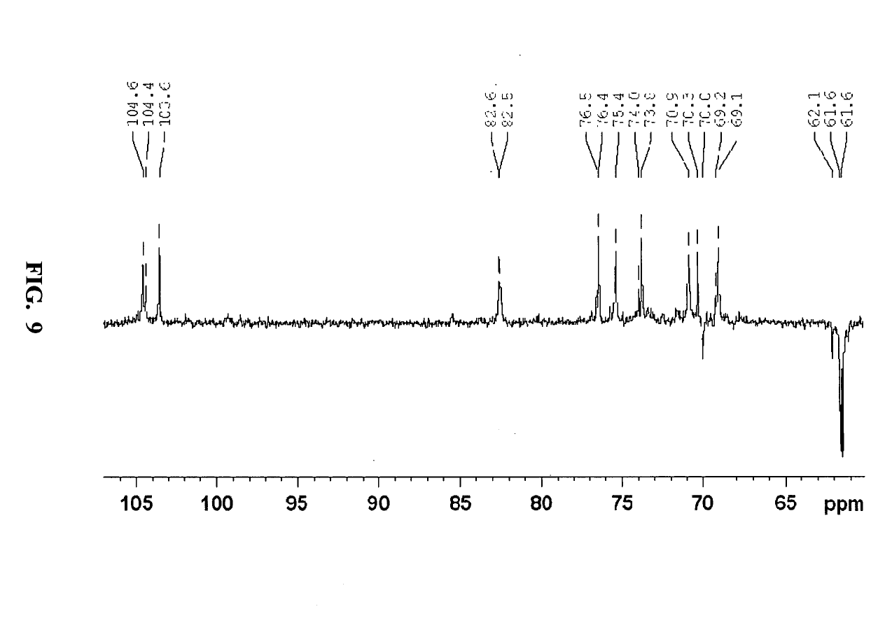

The ¹³C DEPTQ 135 NMR spectrum of fraction A-P-8-deO-deP-1 (FIG. 9) displays one major signal for an anomeric carbon (C-1) at 103.6 ppm and two other anomeric signals close together at 104.6 ppm (major) and 104.4 ppm (minor). The ¹H NMR spectrum at 500 MHz (FIG. 10) displays two sets of anomeric protons (H-1) signals, one centered at 4.52 ppm (d, ³J_{H-1,H-2}˜7.9 Hz, labeled B) and the other one that appeared as a broad doublet of signals centered at 4.73 ppm (labeled A) with a peak separation of approximately 5.5 Hz. In addition to the anomeric signals, the signal at 3.33 ppm was well-separated. Integration of the signals at 4.73 ppm versus the signals at 4.52 or 3.33 ppm gave an approximately 2 to 1 ratio in both the 500 and 800 MHz ¹H NMR spectra. The former signal is attributed to galactose while the latter two are attributed to glucose, indicating that the galactose to glucose molar ratio is 2 to 1. The ¹H NMR spectrum also contained a number of lower intensity signals attributed to impurities (FIG. 10).

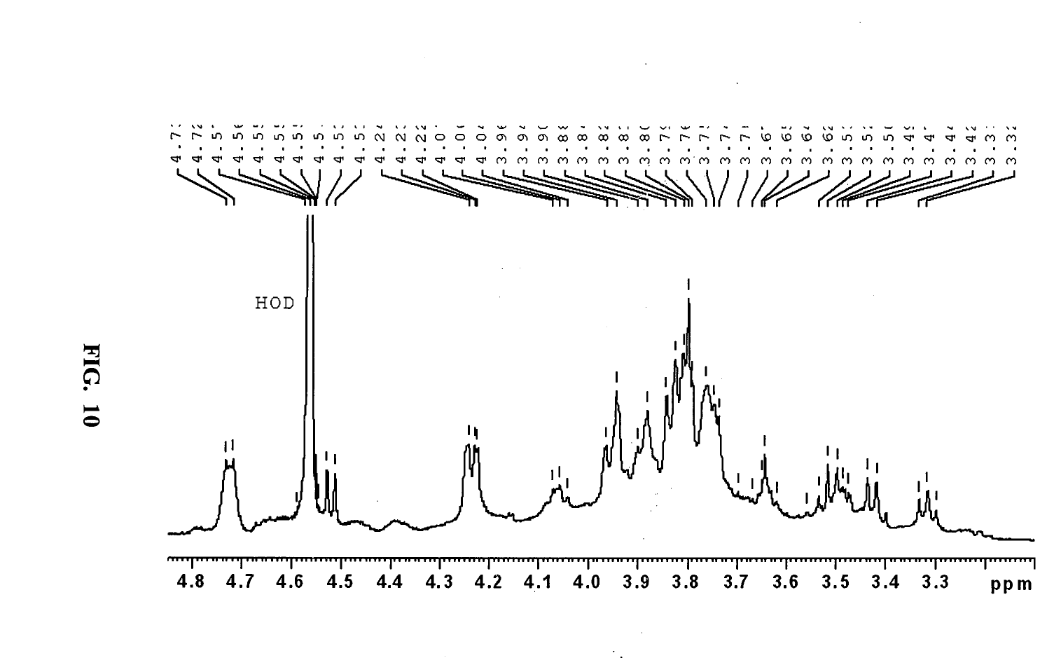

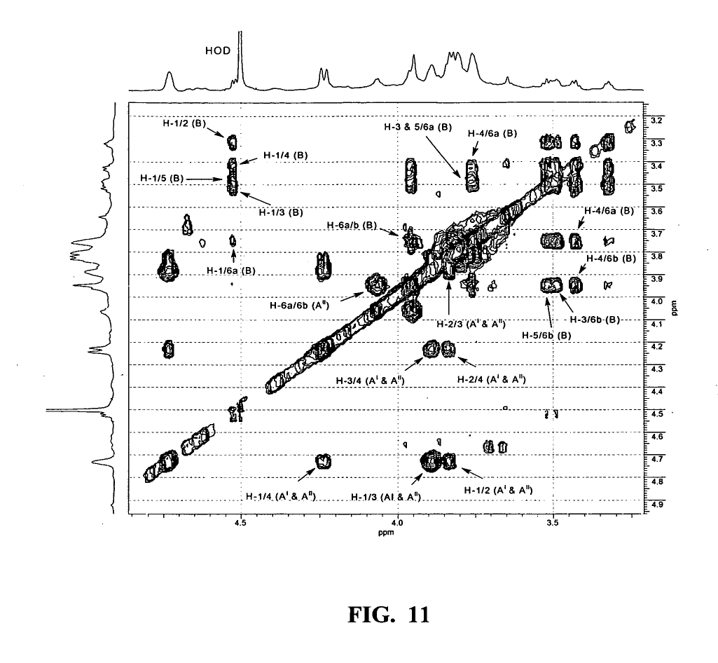

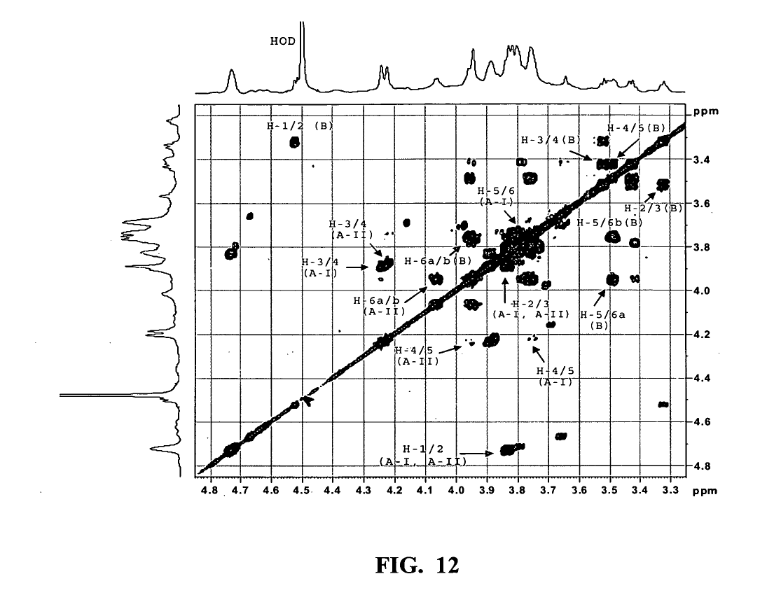

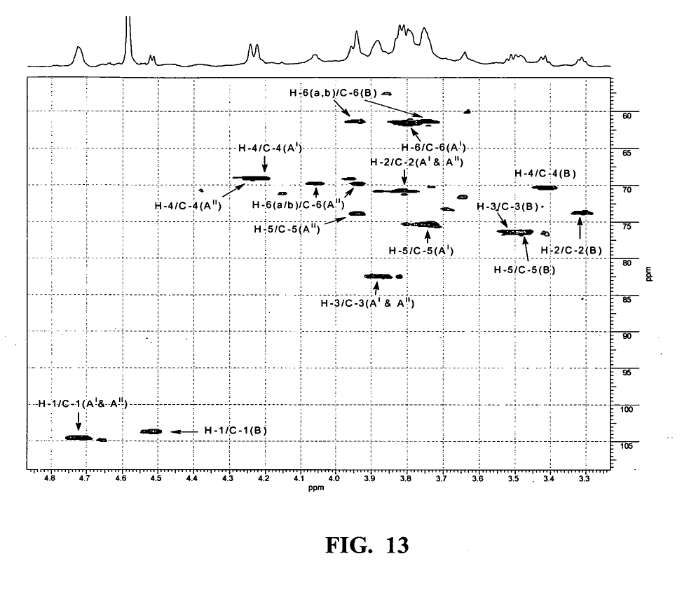

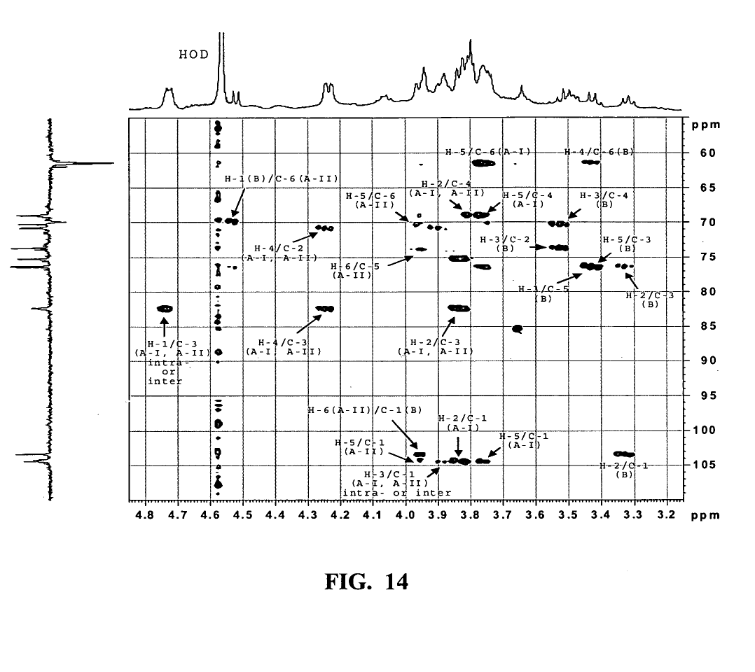

The proton spin system of residue B was fully-identified by tracing connectivities in the TOCSY spectrum at 800 MHz, starting from the H-1 signal at 4.52 ppm to the H-6 signals at 3.77 and 3.97 ppm (FIG. 11), whereas the assignment of the individual ¹H and ¹³C resonances was performed by analysis of the COSY (FIG. 12), ¹H ¹³C HSQC (FIG. 13) and HMBC data (FIG. 14). The large magnitude of the vicinal coupling constants in the range 8 to 10 Hz (Table 3) for all the ring protons is indicative of a β-glucopyranosyl configuration for residue B. The assignment of the anomeric configuration as β was confirmed by the small magnitude of the ¹J_{c-1,H-1} coupling of 163 Hz, measured from the C-1 signal at 103.6 ppm in a ¹³C coupled HSQC experiment, which is typical of axially oriented anomeric (H-1) protons. This β-glucopyranosyl residue was assigned as an unsubstituted branching residue based upon the agreement between the values for all the ¹³C NMR chemical shifts of the monosaccharide (Table 3) with the values reported for unsubstituted methyl β-glucopyranoside.

The anomeric configuration of the galactoses that have H-1 signals centered at 4.73 ppm was assigned as β from the ¹J_{C-1,H-1} coupling of 163 Hz, measured from the C-1 signals at 104.6 and 104.4 ppm in the ¹³C coupled HSQC experiment. In the COSY spectrum at 800 MHz (FIG. 12), this set of H-1 signals showed a cross peak with the signal at 3.83 ppm (t, J˜9.1) that was assigned to H-2 (C-2 at 70.9 ppm from the HSQC spectrum). The latter signal displayed a cross peak with the signal at 3.89 ppm (t, J˜9.4), assigned to H-3 (C-3s at 82.5 and 82.6 ppm from the HSQC spectrum). Cross correlation analysis from the H-3 signal at 3.89 ppm in the COSY spectrum (FIG. 12) revealed the presence of two H-4 signals (C-4-s at 69.1 ppm from the HSQC spectrum) that appear both as doublets (³J_{H-3,H-4}˜2.5 Hz) in the 500 MHz ¹H NMR spectrum in a cold probe (FIG. 10), one at 4.23 ppm and the other one at 4.26 ppm.

On the basis of the comparison of the ¹H/¹³C chemical shifts with those of reference methyl hexopyranosides, the large magnitude of the vicinal coupling constants for H-2 and H-3 in the range 9 to 9.5 Hz, the small magnitude of the ³J_{H-3,H-4} couplings of 2.5 Hz each, and the deshielding of the C-3 signals at 82.5 and 82.6 ppm (+8.7 ppm) with respect of that of methyl β-galactopyranoside, the residues with the H-1 signal centered at 4.73 ppm were assigned to 1,3-linked β-galactopyranosyl residues. These results indicate that the polysaccharide backbone consists of repeating 1,3-linked β-galactopyranosyl units with unsubstituted β-glucopyranosyl branches at positions that have not been discussed as yet.

In the COSY spectrum at 800 MHz (FIG. 12), the H-4 signal at 4.23 ppm appears to have a cross peak with the signal at 3.76 ppm, assigned to H-5 (C-5 at 75.4 ppm from the HSQC spectrum). From cross correlation analysis in the COSY spectrum and inspection of the HSQC spectrum (FIG. 13), the ¹H/¹³C pair at 3.81/61.6 ppm was assigned to H-6/C-6. This residue was named A^{I} and is a 6-unsubstituted 1,3-linked galactose unit.

On the other hand, the H-4 signal at 4.26 ppm displayed a cross peak with the signal at 3.96 ppm (assigned to H-5). The deshielding of this H-5 (+0.2 ppm) with respect to the value of the H-5 for unit A^{I}, suggests that this galactose residue is likely substituted at position 6. In the HSQC spectrum, correlations from the H-5 signal at 3.96 ppm to two ¹³C signals were found, one to 74.2 ppm, assigned to C-5, and the other one to 70.0 ppm, assigned to C-6 (negative in the ¹³C DEPTQ 135 NMR spectrum). The C-6 signal at 70.0 ppm also correlates in the HSQC spectrum (FIG. 13) with the proton signal at 4.07 ppm that in turns correlates in the COSY spectrum with the signal at 3.96 ppm. This latter crosspeak can arise from either a geminal coupling between the two H-6s or from a vicinal coupling of H-6 to H-5. This residue was assigned to a 1,3,6-linked galactose unit, on the basis of the deshielding of the C-6 signal (+8.0 ppm) with respect to that of methyl β-galactopyranoside, and was named A^{II}.

The assignment of the ¹H and ¹³C chemical shifts of the galactoses A^{I} and A^{II} and the glucose residue (B) was confirmed by analysis of the HMBC spectrum (FIG. 14), recorded at 800 MHz with a 60 ms mixing time. From the C-3 signals of the galactoses at 82.5 and 82.6 ppm, two-bond intraresidue correlations to the H-2s (3.83 ppm) and to the H-4s (4.23 and 4.26 ppm) were observed, as well as a three-bond correlation to the H-1s (4.73 ppm) that could not be assigned definitively to interresidue pathways involving the glycosidic linkages since intraresidue pathways are also possible. From the C-1 signal at 104.6 ppm, a two-bond intraresidue correlation to the H-2s at 3.83 ppm was observed, as well as correlations from three-bond intraresidue pathways to H-5 of residue A^{I} at 3.76 ppm and to H-5 of residue A^{II} at 3.96 ppm. The three-bond correlation to the H-3s at 3.89 ppm was observed but could not be assigned to intra or interesidue pathways.

The spectrum also displayed cross peaks between the H-1 and C-1 signals of the glucose residue at 4.52 and 103.6 ppm, respectively, and the C-6 and H-6 signals of the galactose A^{II} at 70.0 and 3.96 ppm, respectively (FIG. 14). These cross peaks were assigned to interesidue pathways involving these positions and establish that the unsubstituted branching β-glucopyranosyl residue (B) is linked to the polysaccharide backbone via 0-6 branching to the 1,3,6-linked β-galactopyranosyl residue A^{II}.

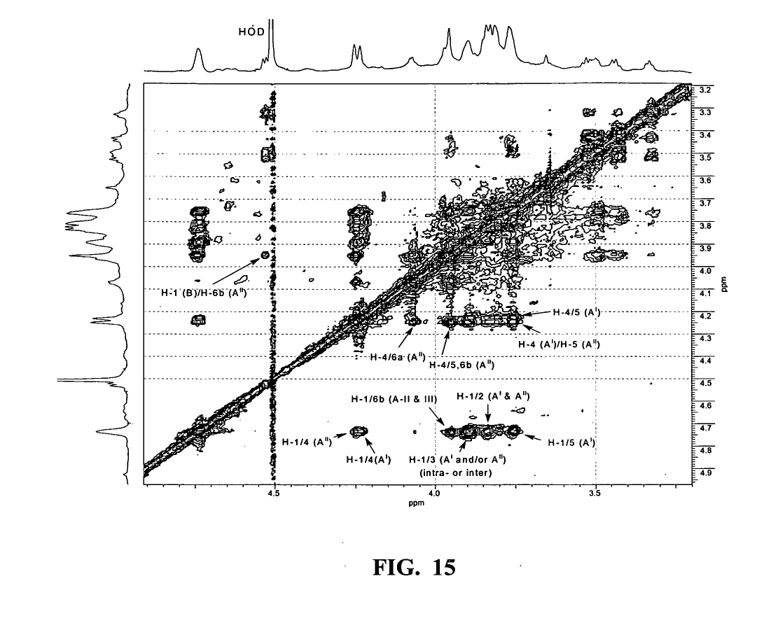

The assignment of the individual residues as unsubstituted branching β-glucopyranosyl and 1,3- and 1,3,6-linked β-galactopyranosyl residues, as well as the linkage pattern was confirmed by recording a NOESY spectrum at 800 MHz with a 200 ms mixing time (FIG. 15). Through-space correlations from the anomeric protons (H-1) to H-3s and H-5s via intraresidue pathways are consistent with the β-anomeric configuration for all the monosaccharide residues. For the galactopyranosyl residues, the H-1/H-3 correlations could also arise from interesidue pathways as a consequence of the β-(1→3)-glycosidic linkages. Meanwhile, a through-space correlation from the H-1 of the unsubstituted branching β-glucopyranosyl residue (B) at 4.52 ppm to the H-6 signal of the 1,3,6-linked β-galactopyranosyl residue A^{II} at 3.96 ppm was unambiguously assigned to the glycosidic linkage that involves these positions (FIG. 15).

TABLE 3
NMR chemical shifts (in ppm) for the galactose residues and the glucose residue of fraction
A-P-8-deO-deP-1 from 2D NMR data recorded at 60° C. in an 800 MHz spectrometer.

| Residue | H/C-1 | H/C-2 | H/C-3 | H/C-4 | H/C-5 | H/C-6 |
|---|---|---|---|---|---|---|
| (A^{I}) | 4.73/ | 3.83 | 3.89 | 4.23/ | 3.76/ | (3.81)/ |
| 3)-β-D-Galp-(1→ | 104.6 | (8.1)/70.9 | (8.4)/82.6 | 69.1 | 75.4 | 61.6 |
| (A^{II}) | 4.73/ | 3.83 | 3.89 | 4.26/ | 3.96/ | (3.96/4.07)/ |
| 3,6)-β-D-Galp-(1→ | 104.6 | (8.1)/70.9 | (8.4)/82.5 | 69.1 | 74.2 | 70.0 |
| (B) | 4.52 | 3.33 | 3.52 | 3.42 | 3.49/ | (3.77/3.97)/ |
| β-D-Glcp-(1→ | (8.0)/103.6 | (8.3)/73.9 | (9.1)/76.4 | (9.0)/70.4 | 76.6 | 61.5 |

Vicinal ³J_{H, H} coupling constants in Hz appear in brackets

On the basis of the 2 to 1 molar ratio of backbone galactosyl units to branching glucosyl units, and the fact that only two galactosyl units are present from preliminary analysis of the NMR data, a simple structure for the dephosphorylated fraction A-P-10-de-O-de-P-1 having a regular alternating substitution of the β-(1→3)-galactan backbone by an unsubstituted β-glucopyranosyl unit at position 6, as shown below.

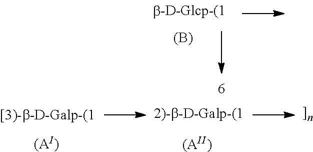

In the absence of a regular alternating substitution, an extreme model considering a random substitution pattern of glucose on galactoses would lead to eight combinations of three galactose units each. In theory, the eight types of galactose units (central galactoses; see below) would have an equal probability to be observed because connection to a 6-substituted galactose from the non-reducing and the reducing end are expected to affect the chemical shifts of a galactose unit (the central one) by different amounts. These eight possibilities are shown following.

### Combinations I to IV (Central 6-Unsubstituted Galactose Residue)

→3)-β-D-Galp-(1→3)-β-D-Galp-(1→3)-β-D-Galp-(1→ I)

central Gal residue connected from the reducing and the non-reducing end to 6-unsubstituted Gals

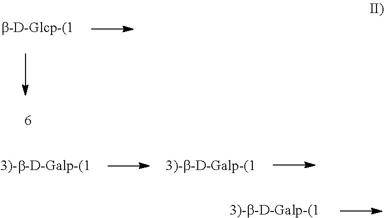

central Gal residue connected from the non-reducing end to a 6-substituted Gal and to a 6-unsubstituted Gal from the reducing end

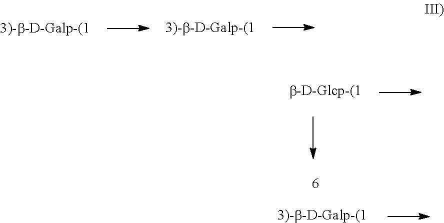

central Gal residue connected from the non-reducing end to a 6-unsubstituted Gal and to a 6-substituted Gal from the reducing end

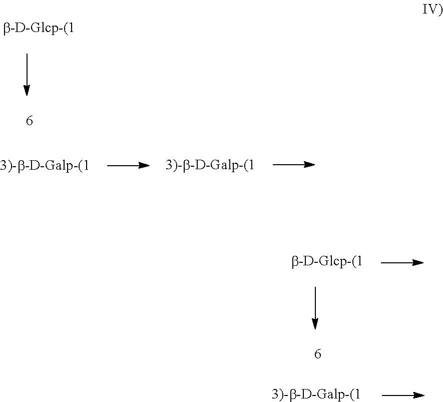

central Gal residue connected from the reducing and the non-reducing end to 6-substituted Gals

### Combinations V to VIII (Central 6-Substituted Galactose Residue)

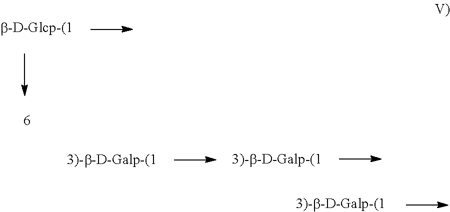

central Gal residue connected from the reducing and the non-reducing end to 6-unsubstituted Gals

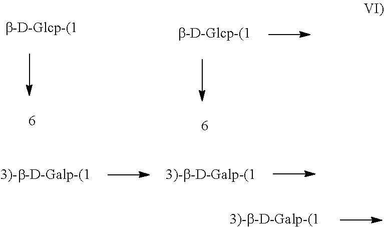

central Gal residue connected from the non-reducing end to a 6-substituted Gal and to a 6-unsubstituted Gal from the reducing end

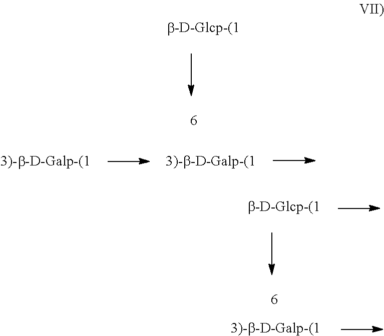

central Gal residue connected from the non-reducing end to a 6-unsubstituted Gal and to a 6-substituted Gal from the reducing end

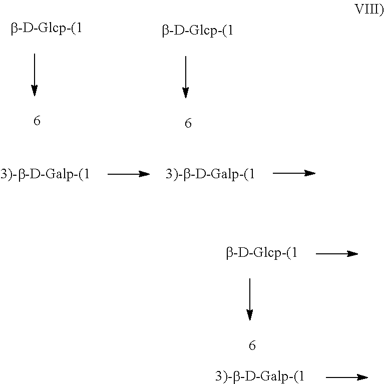

central Gal residue connected from the reducing and the non-reducing end to 6-substituted Gals

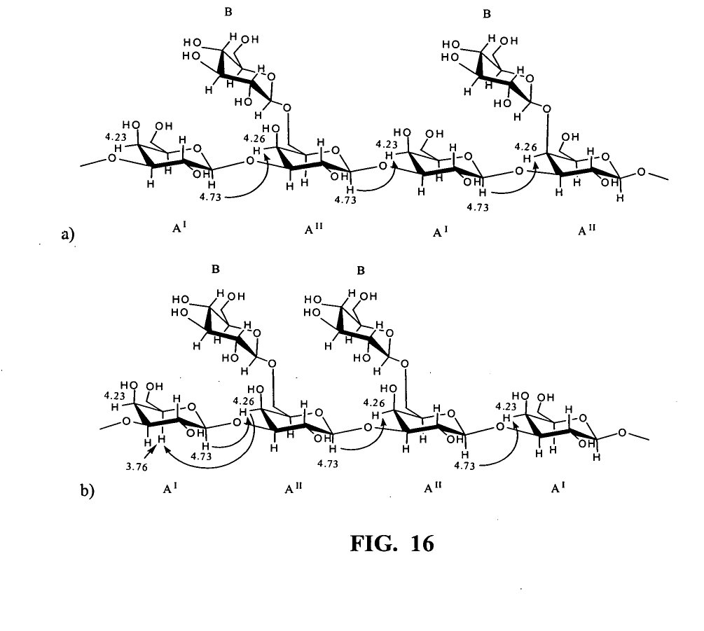

Examination of the NOESY spectrum (FIG. 15), reveals the presence of a cross peak between the signal at 4.26 ppm (H-4 of A^{II}) and the signal at 3.76 ppm (H-5 of A^{I}) that was assigned to an interesidue pathway involving these positions. This establishes that the 1,3,6-linked galactose unit A^{II} is connected from the non-reducing end to the 6-unsubstituted 1,3-linked galactose unit A^{I}. Correlations from interesidue pathways involving the H-4 signals at 4.23 and 4.26 ppm of units A^{I} and A^{II}, respectively, would had served to establish the predominant type of substitution pattern, but given that both A^{I} and A^{II} have H-1 signals at 4.73 ppm, the assignment of the 4.26/4.73 ppm and 4.23/4.73 ppm correlations (FIG. 15) could not be made unambiguously (FIG. 16). For this reason, the correct sequence of units A^{I} and A^{II} could not be made unambiguously and both organized and random structures are possible at this stage in the analysis.

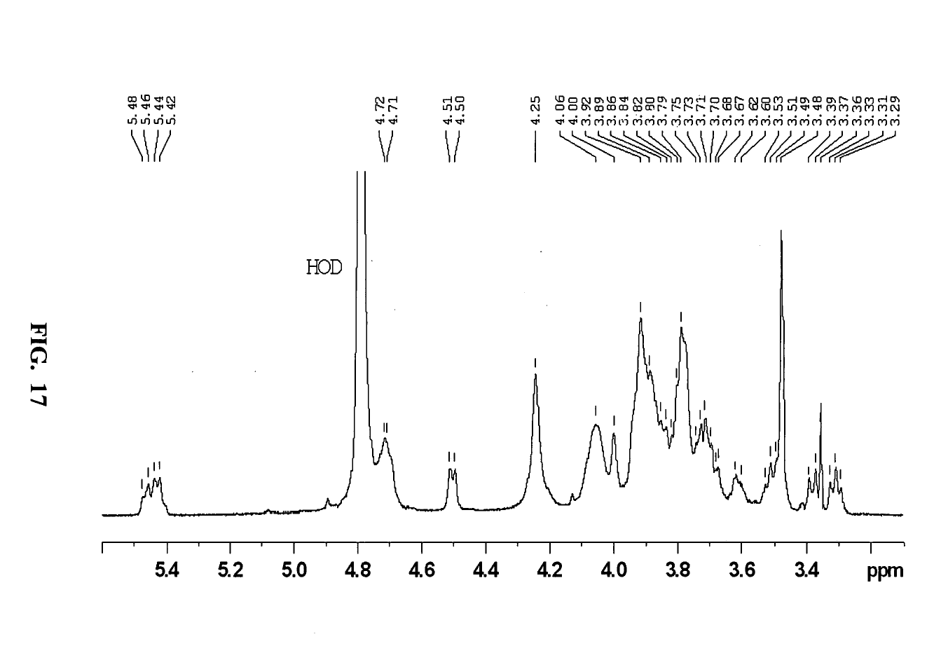

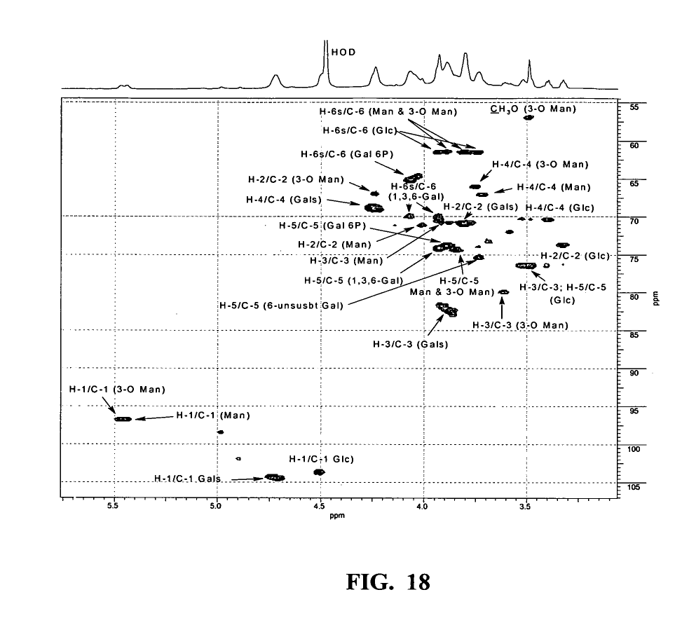

The structure assignment of fraction A-P-8-deO (the whole polysaccharide) and the linkage pattern of the two types of mannose residues and the phosphorylation position(s) in the galactan backbone is determined as follows. First, all the ¹³C and ¹H NMR signals associated with the two types of mannose residues (listed in Table 4) in fraction A-P-8-deO were straightforwardly and unequivocally assigned by analysis of the 2D NMR spectral data (FIGS. 18-21), starting from the C-1/H-1 pairs at 96.8/5.46 and 96.8/5.44 ppm. The magnitude of the anomeric (H-1) chemical shifts at 5.46 (d, J˜8 Hz) and 5.44 ppm (d, J˜8 Hz) (FIG. 17) are consistent with an α anomeric configuration (5.05 and 4.77 ppm for methyl α- and β-mannopyranosides, respectively) for the two types of mannose residues, confirmed by the of ¹J_{C-1,H-1} of 173 Hz observed in the ¹³C coupled HSQC experiment. The peak separations observed for the H-1 signals at 5.46 and 5.44 (8 Hz in each case) in the ¹H NMR spectrum (FIG. 17) are too large to be due to ³J_{H,H} considering that the ³J_{H-1,H-2} coupling in an α mannopyranose is approximately 1.8 Hz, but are of magnitudes consistent with the ³J_{H,P} coupling of 8.5 Hz that was observed in the ¹H NMR spectrum of the reference monosaccharide derivative α-D-mannopyranose 1-phosphate. The ¹J_{c-1,H-}1 value for the reference monosaccharide was 171 Hz. These observations suggest that the two types of mannose residues are phosphorylated at their anomeric (O-1) positions.

In agreement with this observation is the fact that the C-1 chemical shifts at 96.8 ppm (2C) (FIG. 5) are shielded by 5.1 ppm compared to the value of methyl α-D-mannopyranoside at 101.9 ppm, and are deshielded by 1.8 ppm with respect to the value in α-D-mannopyranose at 95.0 ppm. This indicates that in the two types of mannose residues the exo-substitutent located on the β position to the anomeric center is not a carbon atom as in typical glycosidic linkage, but instead is a phosphorous atom from a phosphate group, in agreement with the previous conclusion.

The linkage pattern of the two types of mannose residues was elucidated by recording a ¹H ³¹P HSQC NMR spectrum with an evolution delay adjusted to 8 Hz (FIG. 22). The spectrum displayed strong correlations between the H-1s and H-2s of both α-D-mannose (5.44 and 4.00 ppm, respectively) and 3-O-methyl-α-mannose (5.46 and 4.24 ppm, respectively) and the ³¹P signal at −2.1 ppm, as a result of intraresidue three and four bond couplings, respectively. These assignments were consistent with the observation of ³ J_{H-1,P} and ⁴J_{h-2,P} couplings in the ¹H ³¹P HSQC spectrum of α-D-mannopyranose 1-phosphate. In agreement, the C-2 resonances of α-D-mannose and 3-O-methyl α-mannose appear both as doublets (J˜7.4 Hz) in the ¹³C DEPTQ 135 NMR spectrum (FIG. 5) as a result of ³J_{c-2,P} intraresidue coupling, a pattern also displayed in the ¹³C DEPTQ 135 NMR spectrum of α-D-mannopyranose 1-phosphate. Phosphorylation on O-2 of each type of mannose residue was considered but this possibility was rejected on the basis that it would have resulted in the splitting into doublets of the C-3 signals of both types of mannose residues, and this pattern was not observed in the ¹³C DEPTQ 135 NMR spectrum (FIG. 5). This evidence indicates that α-D-mannose and 3-O-methyl-α-mannose occur both in the form of unsubstituted branching monosaccharides that are linked to the polysaccharide backbone from their anomeric positions via phosphodiester groups.

The phosphorylation position(s) on the galactan backbone were also assigned by analysis of the ¹H ³¹P HSQC spectrum of fraction A-P-8-deO(FIG. 22). The spectrum displayed a correlation between the ³¹P signal al −2.1 ppm and the proton signal at 4.06 ppm that was assigned to an interesidue coupling and suggests that this is the position that is phosphorylated in the polysaccharide. This latter proton signal correlates in the ¹H ¹³C HSQC spectrum (FIG. 18) with the hydroxymethyl ¹³C signal at 65.4 ppm (negative in the ¹³C DEPTQ 135 NMR spectrum, indicating that the phosphorylation occurs on position 6. On the basis that the two types of mannose residues are unsubstituted at position 6, the phosphorylated ¹³C signal at 65.4 was assigned to C-6s of either galactosyl residues from the galactan backbone or glucosyl residues from the side chains. How this assignment was made will be described following.

Phosphorylation at position 6 is corroborated by the absence of the C-6 signal at 65.4 ppm in the ¹³C DEPTQ 135 NMR spectrum (FIG. 9) of the resulting high molecular weight fraction on de-phosphorylation, A-P-10-de-O-de-P-1. The carbons that bear the phosphate groups (65.4 ppm) in the polysaccharide before de-phosphorylation appear in the ¹³C DEPTQ 135 NMR spectrum of fraction A-P-10-de-O-de-P-1 at a more shielded value (61.4 to 61.6 ppm, FIG. 9), characteristic of unsubstituted C-6s of both β-gluco- and β-galactopyranosyl residues.

By inspection of the 2D NMR data of fraction A-P-8-deO (FIGS. 18-21), it was inferred that the glucosyl side chains were unsubstituted at position 6 since all the chemical shifts (see Table 4) are similar to those of the unsubstituted branching β-D-glucopyranosyl unit (B) in fraction A-P-8-deO-deP-1 (listed in Table 3), indicating that the C-6 signal bearing the phosphate groups at 65.4 ppm arises from inner 1,3-linked galactose residues in the polysaccharide backbone. The chemical shift values at 65.4/4.06 ppm are in agreement with the values reported for the C-6/H-6 pair of 6-phosphorylated β-D-galactopyranosyl units in lipopolysaccharides of three types of bacteria.

For instance, Senchenkova (Senchenkova, S. N., et al., Carbohydrate Research (2004), 339, 1342 1347-52) found that in a LPS of Proteus mirabilis, a 1,3-linked β-D-galactopyranosyl unit bearing an ethanolamine phosphate substitutent at O-6, the chemical shifts of C-6 and H-6s were 65.7 and 4.05 ppm, respectively. In a report by Kübler-Kielb, J., et al. (see, Carbohydrate Research (2006), Carbohydrate Research 2006 361, 2980-85) on the structural investigation of a LPS from the Gram-negative bacteria Proteus vulgaris O-34, a β-D-galactopyranosyl unit linked from the reducing end to O-3 of a β-D-galactosamine unit was phosphorylated at O-6 and the chemical shifts for C-6 and H-6s were 65.5 and 4.05 ppm, respectively. In another study, Perepelov (Perepelov, A. V., et al., Carbohydrate Research (2004), 339, 2145-49) and coworkers reported that a β-D-galactopyranosyl unit of the same type described by Kübler-Kielb but also branched at O-2 by a β-D-glucopyranosyl unit had C-6/H-6s at 65.3/4.00 ppm.

On the basis that the ratio of glucose residues (equals the number of 1,3,6-linked galactoses) to the sum of the two types of mannose residues (equals the number of 1,3-linked 6P galactoses) is approximately 1 to 1, most of the 6-unsubstituted 1,3-linked galactoses A^{I} of fraction A-P-8-deO-deP-1 are now substituted at O-6 by α-mannopyranosyl 1-phosphates and the ratio of 1,3,6-linked to 1,3-linked 6P galactoses is approximately 1 to 1. This ratio could be determined from integration of the H-6 signals at 4.08 and 4.06 ppm, respectively, but this could not be done in this way because these signals are overlapped in both the 500 MHz and the 800 MHz ¹H NMR spectra (FIG. 17).

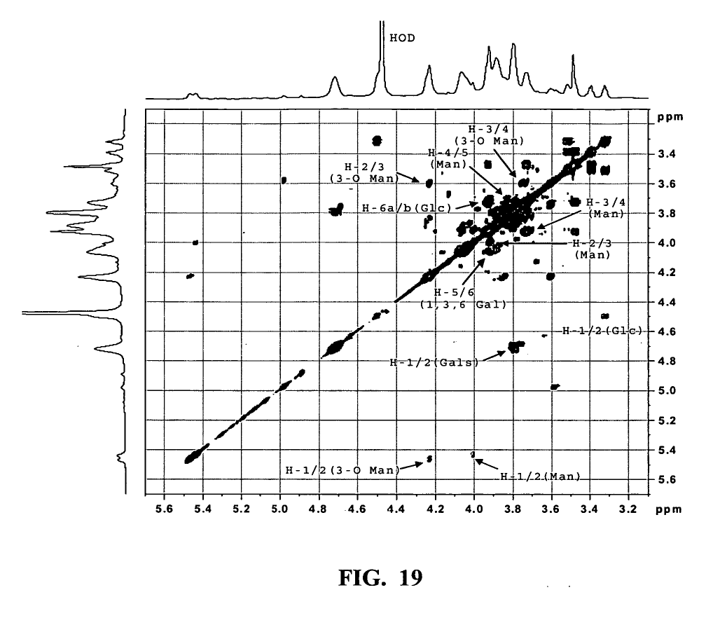

Analysis of the COSY spectrum at 800 MHz (FIG. 19), starting by cross correlation analysis from the H-1s of the β-galactosyl residues in the region of 4.68 to 4.75 ppm indicated the presence of at least four H-1 signals at 4.69 (G^{I}), 4.71 (G^{II}), 4.72 (G^{III}) and 4.74 ppm (G^{IV}), with H-2 signals at 3.82, 3.82, 3.81 and 3.80 ppm, respectively. The assignment of the H-3 signals of the individual galactoses in the region of 3.90 ppm was difficult due to the fact that the crosspeaks from the geminal coupling between the H-6s of the two types of mannose residues at 3.80/3.90 ppm also appear in this region (FIG. 19). By inspection of the region of the crosspeaks between the H-3s and the H-4-s of the galactoses, the latter in the range of 4.23 to 4.26 ppm, four H-3 signals were detected at 3.84 (C-3 at 82.4 ppm from HSQC), 3.87 (C-3 at 83.0 ppm from HSQC), 3.88 (C-3 at 82.5 ppm from HSQC) and 3.91 (C-3 at 81.8 ppm from HSQC), but they could not be unambiguously assigned to the galactoses G^{I}, G^{II}, G^{III} or G^{IV} for the reasons mentioned above.

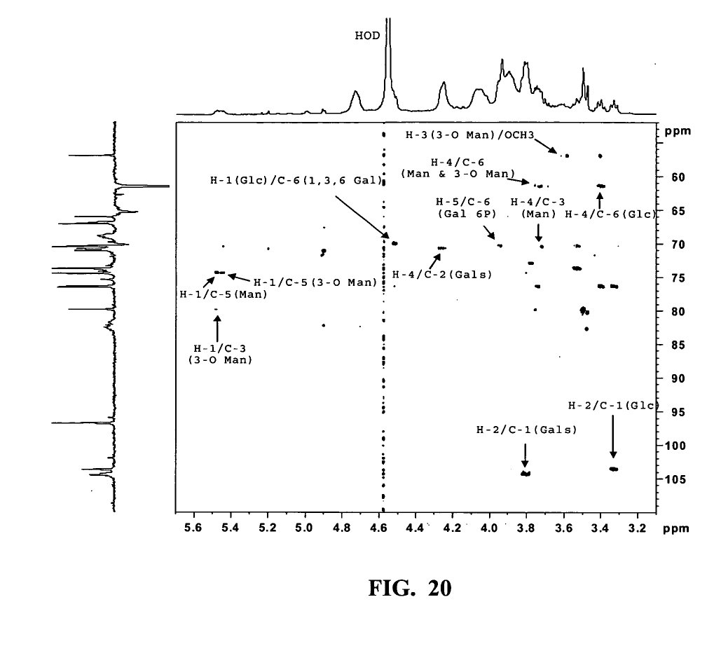

From the ¹H ¹³C HSQC spectrum (FIG. 18) the chemical shifts for the C-6/H-6s (70.2/4.08, 3.95 ppm) and C-5/H-5 (74.2/3.94 ppm) pairs were assigned to these positions in 1,3,6-linked β-galactosyl residues, in agreement with the values found for residue A^{II} in the de-phosphorylated fraction A-P-10-de-O-de-P-1 (see Table 3). In the same spectrum, a ¹³C/¹H pair at 74.0/3.90 ppm was assigned to the C-5/H-5 pair of a 1,3-linked 6P-β-galactosyl residue, on the basis of the observation of a two-bond intraresidue correlation in the HMBC spectrum (FIG. 20) between the H-5 signal at 3.90 ppm and the C-6 signal at 65.4 ppm, that was found earlier to correspond to a carbon bearing a phosphate group.

For the de-phosphorylated portion of the polysaccharide it was concluded that either the structure had a regular substitution of glucose on alternate galactoses, or the chemical shift effects of substitution on adjacent galactoses on a particular galactose were too small to be observed. The structure of the phosphorylated portion could also have a regular substitution pattern, now of alternating glucoses and α-mannopyranosyl 1-phosphate units to galactoses. On this basis, one could draw a simple structure with an alternating O-6-substitution of glucoses and α-mannopyranosyl 1-phosphate units as shown below.

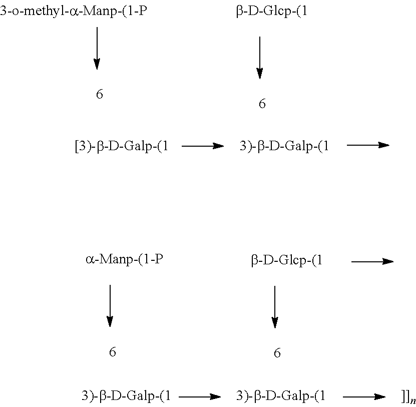

When the methyl group of 3-O-methyl-α-mannopyranosyl 1-phosphate has no effect on the chemical shifts of the 1,3-linked 6P galactoses, a structure of this type yields one type of each 1,3,6-linked and 1,3-linked 6P galactoses in equal amounts. TheThis simple picture described above was not observed; four types of galactose H-1s and at least four types of galactose H-3s were observed.

The observation of more types of galactoses for A-P-10-de-O indicates that substitution by 6-O-glucopyranosyl units and/or 6-O-phosphates diesters does affect the chemical shifts of neighboring galactoses in this more constricted polymer, that is, the second case for the structure of the de-phosphorylated fraction A-P-10-de-O-de-P-1 is correct. An extreme random model leads to exactly the same types of eight combinations of three galactose units considered above, except that now the 6-unsubstituted 1,3-linked galactoses become 1,3-linked 6P galactoses.

A crosspeak is found at 75.5/3.74 ppm was assigned to the C-5/H-5 pair of a 6-unsubstituted 1,3-linked galactose, in agreement with the value found for unit A^{I} in fraction A-P-10-de-O-de-P-1. In the COSY spectrum (FIG. 19), a galactose residue with H-1 and H-2 at 4.69 and 3.78 ppm, respectively, was observed but it could not be assigned to the 6-unsubstituted 1,3-linked galactose residue because the remaining proton signals could not be traced. Nonetheless, this finding is an indication that not all the 6-unsubstituted 1,3-linked galactoses in fraction A-P-8-deO-deP-1 are phosphorylated at O-6 in fraction A-P-8-deO. The presence of this residue is interpreted as an irregularity in the O-6 branching pattern from the eight possible types.

From the NMR evidence it is concluded that the structure is not regularly alternating but could be random or could have some organization (such as blocks) that cannot be determined at this stage and also includes some galactose units that are not substituted at position 6.

TABLE 4
NMR chemical shifts (in ppm) for the two types of mannose residues
and the glucopyranosyl residue of fraction A-P-8-deO.

| Residue | H/C-1 | H/C-2 | H/C-3 | H/C-4 | H/C-5 | H/C-6 |
|---|---|---|---|---|---|---|
| α-Manp-(1- | 5.44^{a}/ | 4.00/ | 3.937 | 3.71/ | 3.84/ | (3.80/3.90)/ |
| P→ | 96.8 | 71.1^{b} | 70.5 | 67.1 | 74.5 | 61.6 |
| 3-O-methyl- | 5.46^{a}/ | 4.24/ | 3.62/ | 3.73/ | 3.85/ | (3.80/3.90)/ |
| α-Manp^{c}-(1- | 96.8 | 66.9^{b} | 79.9 | 66.1 | 74.4 | 61.6 |
| P→ | | | | | | |
| β-D-Glcp- | 4.52 (8.0)/ | 3.33 | 3.52 | 3.42 | 3.49/ | (3.76/3.97)/ |
| (1→ | 103.6 | (8.3)/73.9 | (9.1)/76.4 | (9.0)/70.4 | 76.6 | 61.5 |
| →3,6)-β-D- | (4.68-4.75)/ | (3.80-3.82)/ | (3.84-3.91)/ | (4.23-4.26)/ | 3.94/ | (3.95/4.08)/ |
| Galp-(1→ | (104.4-104.6) | 70.9 | (81.8-83.0) | (68.4-69.2) | 74.2 | 70.2 |
| →3)-β-D- | (4.68-4.75)/ | (3.80-.82)/ | (3.84-3.91)/ | (4.23-4.26)/ | 3.90/ | 4.06/ |
| Galp6P-(1→ | (104.4-104.6) | 70.9 | (81.8-83.0) | (68.4-69.2) | 74.0 | 65.4 |

Vicinal ³J_{H, H} coupling constants in Hz appear in brackets
^{a3}J_{H, P} ~8 Hz;
^{b4}J_{C, P} ~7.4 Hz;
^{c}O—CH₃ at 3.48/57.0 ppm

The structure of the original polysaccharide of fraction A-P-8 differs from that of fraction A-P-8-deO-deP-1 in that the former contains the O-acetyl groups that were removed to facilitate the structural analysis, providing fraction A-P-8-deO.

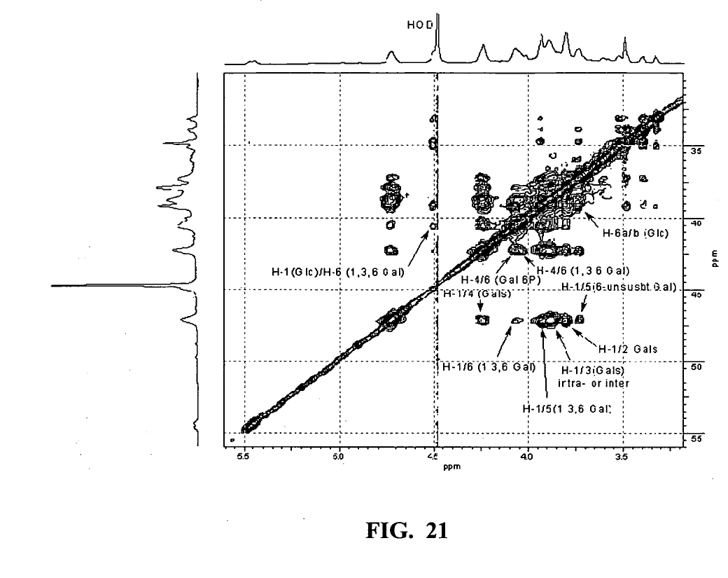

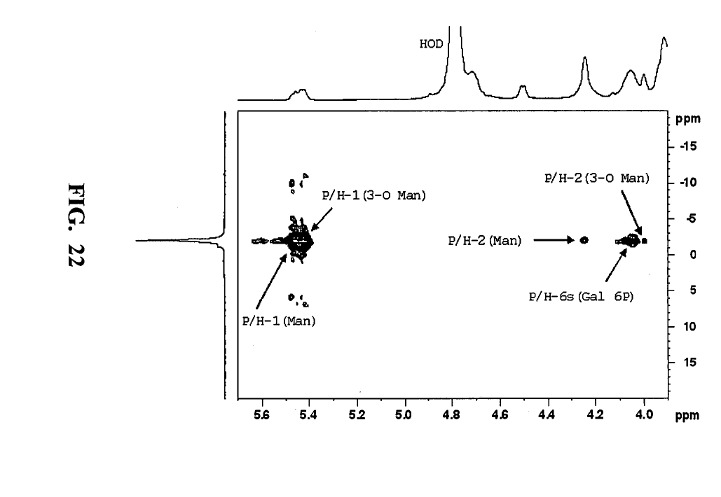

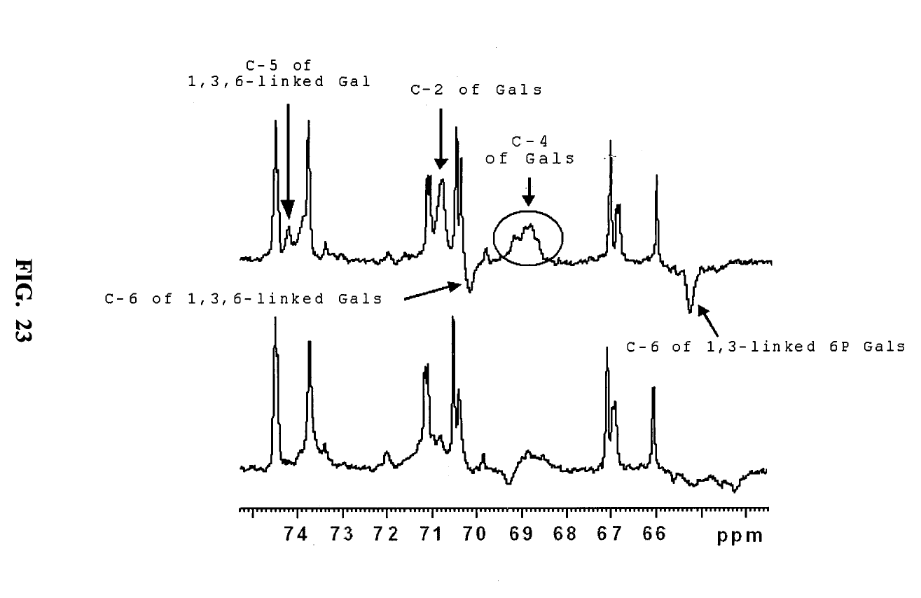

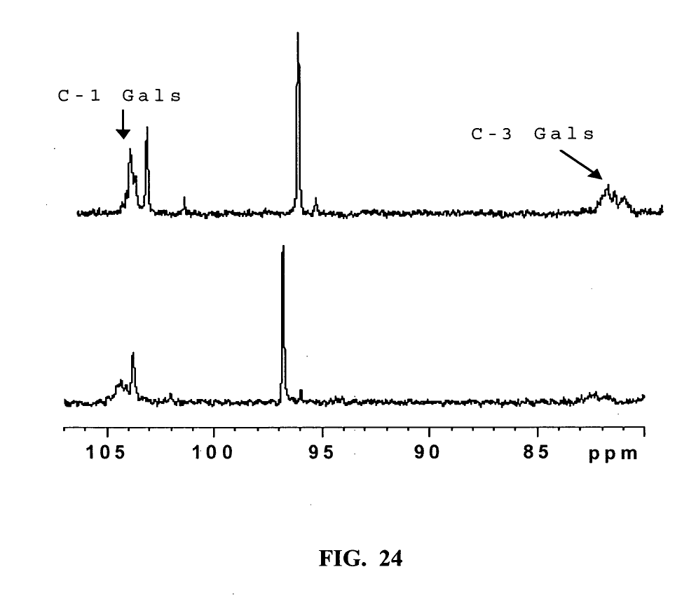

The assignment of the sites of O-acetylation was carried out by comparison of the ¹³C DEPTQ 135 spectra of fractions A-P-8 (before de-O-acetylation) and A-P-8-deO (after de-O-acetylation). In the spectrum of the de-O-acetylated fraction, the most noticeable change corresponds to the C-2 signal of the galactoses at 70.9 ppm that appears as a single peak with intensity comparable to the adjacent mannose C-2 signal at 70.5 ppm, rather than appearing as a broadened peak with one third the peak height (FIG. 23). This is an indication that some galactoses are O-acetylated at O-2. The non-O-acetylated C-2 signals could not be assigned. The effect of acetylation on C-2 on the C-1s of galactoses is clearly observed; before de-O-acetylation the C-1s appear as broad low intensity signals, whereas after de-O-acetylation a narrower signal with double the peak height is observed; similarly the peak heights of the group of signals assigned to C-3 double on de-O-acetylation (FIG. 24).

The signal at 74.2 ppm that was assigned to C-5 of 1,3,6-linked galactose units in the de-O-acetylated fraction A-P-8-deO and does not appear in the ¹³C DEPTQ 135 NMR spectrum of the intact fraction A-P-8 (FIG. 23), most likely as a result of O-acetylation on O-4 of these residues. The presence of O-acetylation on the C-4 position of the galactoses could not be established unambiguously by inspection of the 69.0 ppm region of the C-4 of the galactoses. Nonetheless, the broad signal in this region approximately doubles in peak height and narrows from about 0.8 ppm to about 0.6 ppm on de-O-acetylation, most likely as a result of O-acetylation (FIG. 23).

The peak intensities and shapes of the C-6 signals of the 1,3,6-linked and 1,3-linked 6P galactoses at 70.2 ppm and particularly at 65.4 ppm are affected by removal of the acetyl groups; they become narrower and more intense after de-O-acetylation, most likely as a result of O-acetylation of some extent on O-4s of both types of residues. The low intensity C-6 signals at 65.6, 64.7 and 64.3 ppm in the spectrum of the intact fraction condense to one signal at 65.4 ppm in the de-O-acetylated polysaccharide (FIG. 23).

From the ratio of integrals of the methyl protons of the acetyl groups in the 2.10 ppm region (divided by a factor of 3) to the sum of the anomeric signals of the galactoses in 4.72 ppm region in the 800 MHz ¹H NMR spectrum of fraction A-P-8, it was determined that approximately 35 percent of the galactose units are O-acetylated.

The ¹³C DEPTQ 135 NMR spectra of the remaining fractions (not shown) after anion exchange chromatography of fraction A-P (A-P-3 to A-P-7, A-P-9 to A-P-11) were also recorded. All the spectra displayed a similar pattern of signal positions and intensities, indicating that all the polysaccharides consist of similar repeating units but differ in their molecular sizes.

The results of the immunological testing, measured from the production of nitric oxide by a macrophage cell line on administration of the phosphorylated fractions derived from A-P are shown in FIG. 25. Fractions A-P-1 (mixture of phosphoglycans) and A-P-2 (appears to be a mixture of phosphoglycan-protein complexes), that resulted on size exclusion chromatography on Sephadex G-100 of fraction A-P, are both good immunostimulants, as judged by the fact that in agreement with the crude extract (fraction LW-3-38-1) the nitric oxide levels that were produced on administration of fractions A-P-1 and A-P-2 were above 15 μM for all the doses tested, with the exception of A-P-2 administered at the smaller dose of 1.67 μg/mL.

The immunostimulatory activities of the various phosphoglycan-containing fractions resulting on anion exchange chromatography of fraction A-P-1 are also shown in FIG. 25. The phosphoglycans eluted from the anion exchange column at lower NaCl concentrations (A-P-3 and A-P-5) are poor immunostimulants. The three following phosphoglycan fractions, A-P-6, A-P-7 and A-P-8, only displayed detectable nitric oxide production at the larger dose of 15.0 μg/mL. Of these three fractions, A-P-8 that was eluted from the anion exchange column using the larger NaCl concentration (0.3 M) was most active since it displayed the larger nitric oxide value at the larger dose in comparison to the other two fractions. The phosphoglycan of fraction A-P-9 was eluted from the anion exchange column following that of fraction A-P-8 also using 0.3 M NaCl. Unlike the latter fraction, the phosphoglycan of fraction A-P-9 induced the production of nitric oxide at the middle dose administered (5.0 μg/mL), indicating that it is a better immunostimulant (FIG. 25). On the other hand, fractions A-P-10 and A-P-11 that contain phosphoglycans that were eluted from the anion exchange column using 0.35 and 0.4 M NaCl, respectively, displayed the greatest immunostimulatory activities of all the fractions tested and the results were comparable to those observed for the crude extract (fraction LW-3-38-1) (FIG. 25). From these results, a correlation between the ionic strength of the mobile phase used to detach the phosphoglycans from the anion exchange matrix and their ability to induce nitric oxide production in macrophages is clearly observed.

The ratio of mannosyl phosphate units to glucosyl and galactosyl units is approximately the same for all fractions (integration of the corresponding peaks in the ¹H NMR spectra), and it indicates that the phosphoglycan fractions differ in their sizes (and therefore in the number of anionic mannosyl phosphate chains), which explains their different affinities for the anion exchange matrix and the differences in immunostimulatory activities.

The immunological testing on a de-phosphorylated version of the phosphoglycan of fraction A-P-8 (fraction A-P-8-deO-deP), which displayed nitric oxide production only at the highest dose tested, reflected that the neutral polymeric fragment that was obtained on de-phosphorylation lacked immunostimulatory activity (FIG. 25), indicating that the α-mannopyranosyl 1-phosphate side chains are crucial for the immunostimulatory activity of these phosphoglycans.

Disclosed herein are methods for providing the compositions disclosed herein including the steps of providing a Chlorella extract, contacting the extract with ethanol (e.g. 95% ethanol) to provide a precipitate, contacting the precipitate with an aqueous surfactant (e.g., a quaternary ammonium surfactant) and isolating an insoluble fraction, and size fractionating (e.g., by chromatography ultrafiltration, and/or ion exchange chromatography) the insoluble fraction by using a molecular weight fractionation range of from about 1×10³ to about 1×10⁵ Da, thereby providing the polysaccharide or polysaccharide complex. The precipitate obtained from such a method can be decolorized (e.g., by contacting the precipitate with 2-chloroethanol). Methods can further comprise the steps of suspending Chlorella cells in aqueous media at a temperature of at least about 80° C. followed by centrifuging the media to produce a sediment and a supernatant, and thereafter concentrating the supernatant to provide the Chlorella extract.

Compositions obtained from the methods disclosed herein are also disclosed. Specifically disclosed are compositions comprising a polysaccharide or polysaccharide complex obtained from Chlorella, wherein the polysaccharide or polysaccharide complex has a molecular weight of from about 1×10³ to about 1×10⁵ Daltons. The disclosed compositions can comprise a polysaccharide or polysaccharide complex comprising a phosphorylated-3-O-methyl-mannose residue and/or a phosphorylated D-mannose residue. In some aspects, the polysaccharide or polysaccharide complex can be substantially free of sulfation and/or uronic acid residues.

In some aspects, the polysaccharide or polysaccharide complex comprises repeating units of β-galactopyranosyl linked at O-3. In further aspects, the polysaccharide or polysaccharide complex comprises two 3-linked β-D-galactopyranosyl units phosphorylated at positions O-6 and two 3-linked β-D-galactopyranosyl units glycosylated (branched) at positions O-6. A polysaccharide or polysaccharide complex, as disclosed herein, can comprise any formulae as shown in FIGS. 26 to 34.

Disclosed herein are also polysaccharides and polysaccharide complexes comprising protein or nucleic acids associated with the polysaccharide or polysaccharide complex.

Compositions disclosed herein can also be used as a nutritional supplement comprising a polysaccharide or polysaccharide complex and other substances (e.g., one or more of a carbohydrate, fat, nitrogen source, or mixture thereof). Contemplated nutritional supplements can also comprise one or more supplements, such as vitamin E, vitamin C, vitamin B, folic acid, or a mixture thereof. Nutritional supplements, as contemplated herein, can further comprise one or more other components, such as fish oil, algal oil, fungal oil, marine oil, Spirulina, Echinacea, or a mixture thereof.

Pharmaceutical formulations can comprise a polysaccharide or polysaccharide complex obtained from Chlorella and a pharmaceutically acceptable carrier. Disclosed are methods of modulating an immune response in a subject, comprising the step of administering to the subject an effective amount of any composition disclosed herein, any nutritional supplement disclosed herein, or any pharmaceutical composition or formulation disclosed herein. Also disclosed are methods of treating bacterial or fungal infections in a subject comprising the step of administering to the subject an effective amount of any composition disclosed herein, any nutritional supplement disclosed herein, or any pharmaceutical composition or formulation disclosed herein. Disclosed are also methods of vaccinating a subject comprising the step of administering to the subject a vaccine and an effective amount of any composition disclosed herein, any nutritional supplement disclosed herein, or any pharmaceutical composition or formulation disclosed herein.

Further disclosed herein is the use of a polysaccharide or polysaccharide complex as disclosed herein for the use in making a medicament for modulating an immune response in a subject.

Yet further disclosed herein is the use of a polysaccharide or polysaccharide complex as disclosed herein for the use in making a medicament for treating bacterial or fungal infections in a subject.

Still further disclosed herein is the use of a polysaccharide or polysaccharide complex as disclosed herein for the use in making a medicament for vaccinating a subject.

It will be apparent to those skilled in the art that various modifications and variations can be made in the present disclosure without departing from the scope or spirit of the present disclosure. Other embodiments of the disclosure will be apparent to those skilled in the art from consideration of the specification and practice of the subject matter disclosed herein. It is intended that the specification and examples be considered as exemplary only, with a true scope and spirit of the disclosure being indicated by the following claims.

1. A polysaccharide or polysaccharide complex obtained from Chlorella, comprising:
i) at least one methylated phosphosaccharide unit having the formula:
3-O-methyl α-Manp-(1-PO₃H→; or
ii) at least one phosphosaccharide unit having the formula:
α-D-Manp-(1-PO₃H→;
wherein the polysaccharide or polysaccharide complex has a molecular weight of from about 1×10³ to about 1×10⁶ Da.

2. The polysaccharide or polysaccharide complex of claim 1, wherein the polysaccharide or polysaccharide complex is substantially free of sulfation.

3. The polysaccharide or polysaccharide complex of claim 1, wherein the polysaccharide or polysaccharide complex is substantially free of uronic acid residues.

4. The polysaccharide or polysaccharide complex of claim 1, wherein the polysaccharide or polysaccharide complex comprises repeating units of β-D-galactopyranosyl residues linked at O-3.

5. The polysaccharide or polysaccharide complex of claim 1, wherein the polysaccharide or polysaccharide complex comprises a 6-phosphorylated β-D-galactopyranosyl unit.

6. The polysaccharide or polysaccharide complex of claim 1, wherein the polysaccharide or polysaccharide complex comprises a β-D-glucopyranosyl-(1→6)-β-D-galactopyranosyl-(1→.

7. The polysaccharide or polysaccharide complex of claim 1, comprising a polysaccharide according to FIG. 26.

8. The polysaccharide or polysaccharide complex of claim 1, comprising a polysaccharide according to FIG. 27.

9. The polysaccharide or polysaccharide complex of claim 1, comprising a polysaccharide according to FIG. 28.

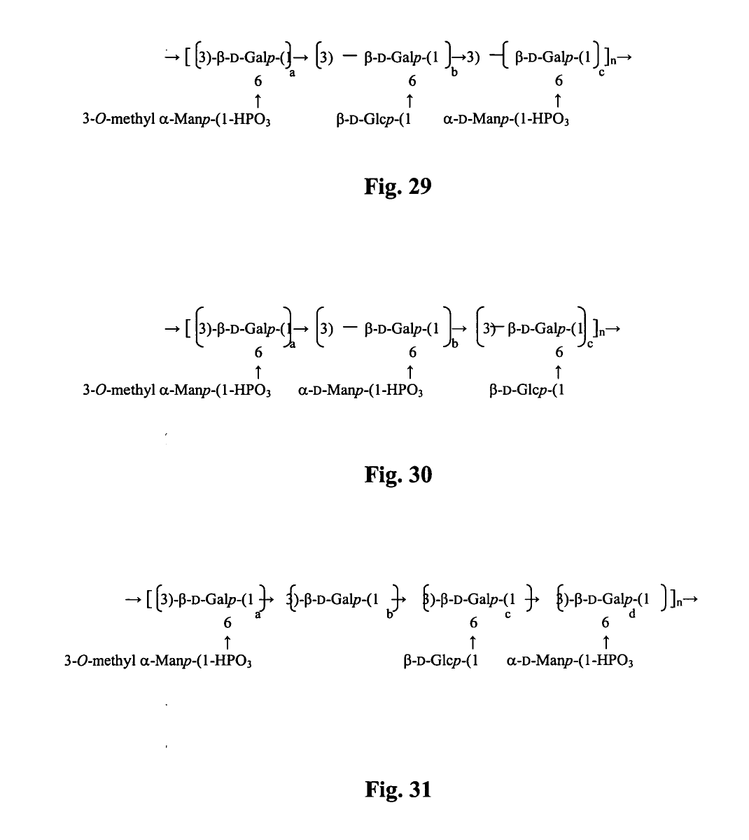

10. The polysaccharide or polysaccharide complex of claim 1, comprising a polysaccharide according to FIG. 29.

11. The polysaccharide or polysaccharide complex of claim 1, comprising a polysaccharide according to FIG. 30.

12. The polysaccharide or polysaccharide complex of claim 1, comprising a polysaccharide according to FIG. 31.

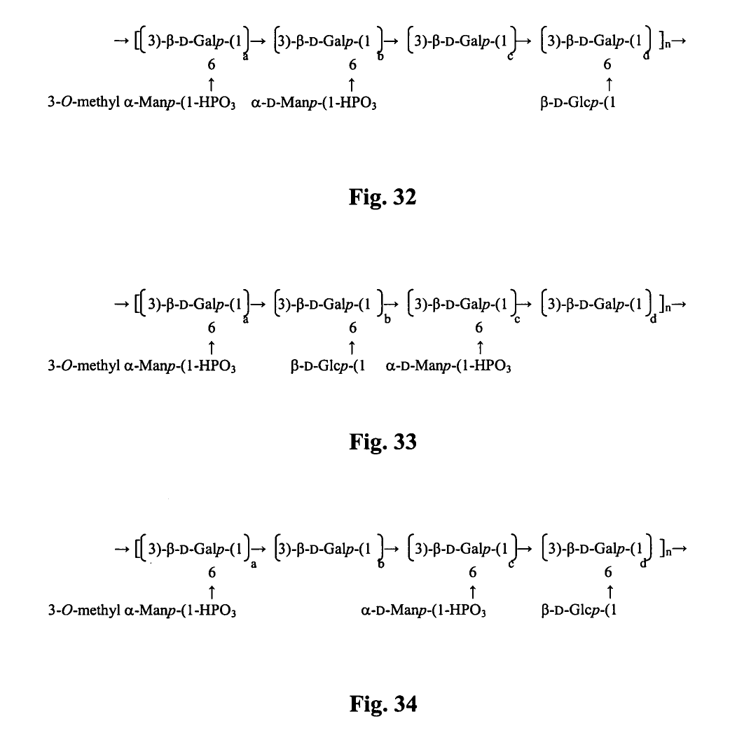

13. The polysaccharide or polysaccharide complex of claim 1, comprising a polysaccharide according to FIG. 32.

14. The polysaccharide or polysaccharide complex of claim 1, comprising a polysaccharide according to FIG. 33.

15. The polysaccharide or polysaccharide complex of claim 1, comprising a polysaccharide according to FIG. 34.

16. The polysaccharide or polysaccharide complex of claim 1, further comprising one or more proteins or nucleic acids that are associated with the polysaccharide or polysaccharide complex.

17. The polysaccharide or polysaccharide complex of claim 1, wherein the polysaccharide or polysaccharide complex are obtained from C. minutissima, C. marina, C. salina, C. vulgaris, C. anitrata, C. antarctica, C. autotrophica, C. regularis, C. ellipsoidea, or from a mixture thereof.

18. The polysaccharide or polysaccharide complex of claim 1, wherein the polysaccharide or polysaccharide complex are obtained from obtained from C. pyrenoidosa.

19-20. (canceled)

21. A supplemental nutritional composition comprising:
a) one or more polysaccharide or polysaccharide complexes, comprising:
i) at least one methylated phosphosaccharide unit having the formula:
3-O-methyl α-Manp-(1-PO₃H→;
wherein the polysaccharide or polysaccharide complex has a molecular weight of from about 1×10³ to about 1×10⁶ Da; and
b) one or more comestible or nutritional ingredients.

22-38. (canceled)

39. The composition according to claim 21, further comprising one or more sources of a carbohydrate, a fat, or nitrogen.

40. The composition according to claim 21, further comprising one or more supplements chosen from vitamin E, vitamin C, vitamin B, and folic acid

41. The composition according claim 21, further comprising one or more oils chosen from fish oil, algal oil, fungal oil, marine oil, Spirulina, and Echinacea.

42. A method for preparing a polysaccharide or polysaccharide complex, comprising:
a) contacting a Chlorella extract with 95% ethanol to form a precipitate;
b) contacting the precipitate with an aqueous solution of a surfactant to form an insoluble fraction and isolating the insoluble fraction;
c) size fractionating the insoluble fraction by using a molecular weight fractionation range of from about 1×10³ to about 1×10⁵ Da; and
d) isolating one or more polysaccharide or polysaccharide complexes, comprising:
i) at least one methylated phosphosaccharide unit having the formula:
3-O-methyl α-Manp-(1-PO₃H→; or
ii) at least one phosphosaccharide unit having the formula:
α-D-Manp-(1-PO₃H→;
wherein the isolated polysaccharide or polysaccharide complex has a molecular weight of from about 1×10³ to about 1×10⁶ Da.

43. The method according to claim 42, wherein after step (b), the precipitate is decolorized.

44. (canceled)

45. The method according to claim 42, wherein the surfactant is a quaternary ammonium compound.

46. (canceled)

47. (canceled)

48. A composition comprising a polysaccharide or polysaccharide complex obtained from Chlorella by the method of claim 42.

49. A method of modulating an immune response in a subject, comprising administering to the subject an effective amount of a polysaccharide or polysaccharide complex obtained from Chlorella, comprising:
i) at least one methylated phosphosaccharide unit having the formula:
3-O-methyl α-Manp-(1-PO₃H→; or
ii) at least one phosphosaccharide unit having the formula:
α-D-Manp-(1-PO₃H→;
wherein the polysaccharide or polysaccharide complex has a molecular weight of from about 1×10³ to about 1×10⁶ Da.

50. A method of treating bacterial or fungal infections in a subject, comprising administering to the subject an effective amount of a polysaccharide or polysaccharide complex obtained from Chlorella, comprising:
i) at least one methylated phosphosaccharide unit having the formula:
3-O-methyl α-Manp-(1-PO₃H→; or
ii) at least one phosphosaccharide unit having the formula:
α-D-Manp-(1-PO₃H→;
wherein the polysaccharide or polysaccharide complex has a molecular weight of from about 1×10³ to about 1×10⁶ Da.

51. A method of vaccinating a subject, comprising administering to the subject a vaccine and an effective amount of a polysaccharide or polysaccharide complex obtained from Chlorella, comprising:
i) at least one methylated phosphosaccharide unit having the formula:
3-O-methyl α-Manp-(1-PO₃H→; or
ii) at least one phosphosaccharide unit having the formula:
α-D-Manp-(1-PO₃H→;
wherein the polysaccharide or polysaccharide complex has a molecular weight of from about 1×10³ to about 1×10⁶ Da.

52-54. (canceled)
